(19) United States
(12) Patent Application Publication
(10) Pub. No.: US 2026/0092120 A1
(43) Pub. Date: Apr. 2, 2026

(54) COMPOSITIONS AND METHODS

(71) Applicant: Flagship Pioneering Innovations VII, LLC, Cambridge, MA (US)

(72) Inventors: Joseph Richard BRADY, Somerville, MA (US); Sabrina Yusang YANG, Cambridge, MA (US); John Daniel MENDLEIN, Scituate, MA (US)

(21) Appl. No.: 18/855,846

(22) PCT Filed: Apr. 11, 2023
(86) PCT No.: PCT/US2023/065640

§ 371 Date: Oct. 10, 2024

Related U.S. Application Data
(60) Provisional application No. 63/329,747, filed on Apr. 11, 2022.
(60) Provisional application No. 63/329,753, filed on Apr. 11, 2022.

(51) Int. Cl. C07K 16/28; C07K 14/475; C07K 14/52; C07K 14/575; C07K 19/00; C12N 15/62

(52) U.S. Cl. CPC C07K 16/2857; C07K 14/475; C07K 14/52; C07K 14/575; C07K 19/00; C12N 15/62; C07K 2319/00

(57) ABSTRACT

Provided herein are macromolecules that conditionally induce a cellular effector function (e.g., a biological or therapeutic activity) based on the presence of a disease signature ligand, compositions comprising the same, and methods of using the same.

### SEQUENCE LISTING

The instant application contains a Sequence Listing which has been submitted electronically in XML format and is hereby incorporated by reference in its entirety. Said XML copy, created on Apr. 11, 2023, is named 51661-002WO2_Sequence_Listing_4_11_23 and is 31,499 bytes bytes in size.

### BACKGROUND

There is a need in the art for macromolecules that conditionally induce a cellular effector function (e.g., a biological or therapeutic activity) based on the presence of a disease signature ligand and for methods of using the same.

### SUMMARY OF THE INVENTION

In one aspect, provided herein is a macromolecule comprising a first binding domain linked to a second binding domain, wherein (a) the first binding domain specifically binds a disease signature ligand in a biological sample; and (b) the second binding domain specifically binds an effector ligand in the biological sample and induces a cellular effector function upon binding to the effector ligand; wherein the macromolecule is capable of forming a multimer in the presence of the disease signature ligand, and wherein induction of the effector function by the macromolecule is conditional upon each member of the multimer binding the disease signature ligand.

In some embodiments, the multimer is a dimer, trimer, or tetramer. In some embodiments, the multimer is a dimer. In some embodiments, the dimer is a homodimer.

In another aspect, provided herein is a pair of macromolecules, each independently comprising a first binding domain linked to a second binding domain, wherein (a) the first binding domain of each macromolecule specifically binds a disease signature ligand in a biological sample; (b) the second binding domain of a first member of the pair of macromolecules specifically binds a first effector ligand in the biological sample; and (c) the second binding domain of a second member of the pair of macromolecules specifically binds a second effector ligand in the biological sample; wherein the second binding domain of the first member of the pair of macromolecules and the second binding domain of the second member of the pair of macromolecules induce a cellular effector function upon binding to the first effector ligand and the second effector ligand; wherein the pair of macromolecules is capable of forming a heteromultimer in the presence of the disease signature ligand, and wherein induction of the effector function by the macromolecule is conditional upon each member of the heteromultimer binding the disease signature ligand.

In another aspect, provided herein is a pair of macromolecules, each independently comprising a first binding domain linked to a second binding domain, wherein (a) the first member of the pair of macromolecules comprises a first binding domain 1 (FBD1) that specifically binds a first epitope of disease signature ligand in a biological sample; (b) the second member of the pair of macromolecules comprises a first binding domain 2 (FBD2) that specifically binds a second epitope of disease signature ligand in a biological sample; and (c) the second binding domain specifically binds an effector ligand in the biological sample and induces a cellular effector function upon binding to the effector ligand; wherein the macromolecule is capable of forming a multimer in the presence of the disease signature ligand, and wherein induction of the effector function by the macromolecule is conditional upon each member of the multimer binding the disease signature ligand.

In another aspect, provided herein is a pair of macromolecules, each independently comprising a first binding domain linked to a second binding domain, wherein (a) the first member of the pair of macromolecules comprises a first binding domain 1 (FBD1) that specifically binds a first epitope of disease signature ligand in a biological sample; (b) the second member of the pair of macromolecules comprises a first binding domain 2 (FBD2) that specifically binds a second epitope of disease signature ligand in a biological sample; (c) the second binding domain of the first member of the pair of macromolecules specifically binds a first effector ligand in the biological sample; and (d) the second binding domain of the second member of the pair of macromolecules specifically binds a second effector ligand in the biological sample; wherein the macromolecule is capable of forming a multimer in the presence of the disease signature ligand, and wherein induction of the effector function by the macromolecule is conditional upon each member of the multimer binding the disease signature ligand.

In certain of the foregoing aspects, the first binding domain and second binding domain are linked covalently (e.g., by small molecule, peptide (e.g., as a single polypeptide chain comprising the first binding domain and second binding domain), or a combination thereof).

In some embodiments, the heteromultimer is a dimer.

In some embodiments of any of the above aspects, the disease signature ligand is a protein, a peptide, or a small molecule. In some embodiments, the protein is a soluble protein or an insoluble protein.

In some embodiments, the disease signature ligand is a cytokine. In some embodiments, the cytokine is an interleukin, an interferon, a growth factor, a chemokine, or a member of the TNF family. In some embodiments, the interleukin is IL-1, IL-2, IL-4, IL-6, IL-8, IL-10, IL-12, IL-15, IL-17, or IL-23; the interferon is IFN-gamma; the growth factor is transforming growth factor beta (TGF-beta), granulocyte colony stimulating factor (GCSF) granulocyte-macrophage colony-stimulating factor (GCSF), epidermal growth factor (EGF), or erythropoietin (EPO); the chemokine is monocyte chemoattractant protein-1 (MCP-1) or interferon gamma-induced protein 10 (IP-10); or the member of the TNF family is TNF-alpha.

In some embodiments, the disease signature ligand is a cell surface receptor, a surface antigen, a membrane-bound protein, an extracellular matrix component, or an integrin.

In some embodiments, the disease signature ligand is a self antigen of an organism from which the biological sample is derived. In some embodiments, the self antigen is an anti-drug antibody (ADA), an autoantibody, or a tumor marker.

In some embodiments, the disease signature ligand is a nucleic acid.

In some embodiments, the disease signature ligand is a carbohydrate, a lipid, a peptide, a nucleoside, or a combination of the foregoing.

In some embodiments, the disease signature ligand is a hormone, an amino acid derivative, a steroid, or an eicosanoid.

In some embodiments, the disease signature ligand is a non-self antigen. In some embodiments, the disease signature ligand is a virus, a bacterium, or a fragment or antigen thereof.

In some embodiments, the disease signature ligand is multimeric. In some embodiments, the disease signature ligand is dimeric, trimeric, or tetrameric.

In some embodiments of any of the above aspects, the first binding domain, FBD1, or FBD2 comprises a polypeptide that specifically binds the disease signature ligand. In some embodiments, the polypeptide is an antibody or a fragment thereof. In some embodiments, the antibody or fragment thereof is an scFv, a monospecific tandem scFv (taFv), a bispecific taFv, a VHH, a VNAR, a Fab, a monospecific single-chain diabody, a bispecific single-chain diabody, or a dual-affinity re-targeting antibody (DART).

In some embodiments, the polypeptide is an antibody mimetic. In some embodiments, the antibody mimetic is an affibody, an affilin, an affimer, an affitin, an alphabody, an anticalin, a lipocalin, an avimer, a DARPin, a fynomer, a gastrobody, a knottin, a Kunitz domain peptide, a monobody, a fibronectin type III domain (FN3)-based binder, a nanoantibody, a nanoCLAMP, an optimer, a repebody, a pronectin, a centyrin, an obody, a peptide aptamer, a synthetic peptide, or a variable lymphocyte receptor (VLR).

In some embodiments, the polypeptide is an endogenous binding domain. In some embodiments, the endogenous binding domain is a cell receptor domain, an enzyme domain, a variable lymphocyte receptor (VLR) domain, a receptor ectodomain, a nuclear hormone receptor ligand-binding domain, or a DNA-binding domain.

In some embodiments, the first binding domain, FBD1, or FBD2 comprises an oligonucleotide that specifically binds the disease signature ligand. In some embodiments, the oligonucleotide is a nucleic acid aptamer. In some embodiments, the nucleic acid aptamer is a DNA aptamer.

In some embodiments, the first binding domain comprises a chemical molecule that specifically binds the disease signature ligand.

In some embodiments, the first binding domain, FBD1, or FBD2 has affinity to two or more disease signature moieties.

In some embodiments of any of the above aspects, the effector ligand is a protein or a peptide.

In some embodiments, the effector ligand is a cell-surface receptor or an intracellular receptor. In some embodiments, the cell-surface receptor is a catalytic receptor or the intracellular receptor is a nuclear hormone receptor.

In some embodiments, the catalytic receptor is a receptor tyrosine kinase (RTK), a receptor serine/threonine kinase (RSK), a type 1 cytokine receptor, a type 2 cytokine receptor, or a tumor necrosis factor (TNF) superfamily receptor. In some embodiments, the RTK is VEGFR, the RSK is TGFBR2, the type 1 cytokine receptor is IL-2R, the type 2 cytokine receptor is IL10R, or the TNF superfamily receptor is TNFR2 or 4-1BB.

In some embodiments, the second binding domain is an agonist of the effector ligand.

In some embodiments, the effector ligand must be homodimerized to exert a cellular effector function. In some embodiments, the effector ligand is homodimerized in the presence of the multimer of the macromolecule.

In some embodiments, the first effector ligand and the second effector ligand must be associated to exert a cellular effector function. In some embodiments, the first effector ligand and the second effector ligand are associated in the presence of the multimer of the macromolecule. In some embodiments, the association is heterodimerization.

In some embodiments of any of the above aspects, the cellular effector function is a biological activity.

In some embodiments, the cellular effector function is a therapeutic activity.

In some embodiments, the cellular effector function is a disease activity.

In some embodiments of any of the above aspects, the second binding domain comprises a polypeptide that specifically binds the effector ligand. In some embodiments, the polypeptide is an antibody or a fragment thereof. In some embodiments, the antibody or fragment thereof is an scFv, a monospecific taFv, a bispecific taFv, a VHH, a VNAR, a Fab, a monospecific single-chain diabody, a bispecific single-chain diabody, or a DART.

In some embodiments, the polypeptide is an antibody mimetic. In some embodiments, the antibody mimetic is an affibody, an affilin, an affimer, an affitin, an alphabody, an anticalin, a lipocalin, an avimer, a DARPin, a fynomer, a gastrobody, a knottin, a Kunitz domain peptide, a monobody, a FN3-based binder, a nanoantibody, a nanoCLAMP, an optimer, a repebody, a pronectin, a centyrin, an obody, a peptide aptamer, a synthetic peptide, or a VLR.

In some embodiments, the polypeptide is an endogenous binding domain. In some embodiments, the endogenous binding domain is a ligand of the effector ligand or a fragment thereof. In some embodiments, the endogenous binding domain is a viral binding protein or a fragment thereof.

In some embodiments, the second binding domain comprises an oligonucleotide that specifically binds the effector ligand. In some embodiments, the oligonucleotide is a nucleic acid aptamer. In some embodiments, the nucleic acid aptamer is a DNA aptamer.

In some embodiments, the first binding domain, FBD1, or FBD2 comprises a chemical molecule that specifically binds the disease signature ligand.

In some embodiments of the aspect comprising a pair of macromolecules, the second binding domain of the first member of the pair of macromolecules is a first portion of a binding moiety and the second binding domain of the second member of the pair of macromolecules is a second portion of the binding moiety.

In some embodiments of the aspects comprising a pair of macromolecules, the second binding domain of the first member of the pair of macromolecules specifically binds to a first component of a heterodimeric receptor and the second binding domain of the second member specifically binds to a second component of the heterodimeric receptor.

In some embodiments of the aspects comprising a pair of macromolecules, the second binding domain of the first member of the pair of macromolecules is a first component of a dimeric moiety and the second binding domain of the second member is a second component of a dimeric moiety.

In some embodiments of the aspects comprising a pair of macromolecules, the second binding domain of the first member of the pair of macromolecules is a first fragment of a polypeptide chain and the second binding domain of the second member is a second fragment of the polypeptide chain. In some embodiments, the polypeptide chain is a hormone, a cytokine, or a growth factor.

In some embodiments of the aspects comprising a pair of macromolecules, the second binding domain of the first member of the pair of macromolecules and the second binding domain of the second member of the pair of macromolecules have been engineered to have reduced affinity for one another.

In some embodiments of any of the above aspects, the macromolecule comprises a reporter moiety. In some embodiments, the reporter moiety is an affinity tag, a fluorescent marker, a radioactive marker, or a chromogenic marker. In some embodiments, the affinity tag is a FLAG affinity tag or the chromogenic marker is luciferase or beta-lactamase.

In some embodiments of any of the above aspects, the macromolecule or pair of macromolecules further comprises one or more linker domains. In some embodiments, the one or more linker domains are peptide linkers. In some embodiments, the peptide linkers comprise GS linkers. In some embodiments, the GS linkers comprise GS(GₙS)ₘ linkers. In some embodiments, the GS linkers comprise (GₙS)ₘ linkers.

In some embodiments of any of the above aspects, the macromolecule is a polypeptide.

In some embodiments of any of the above aspects, the biological sample is an extract, fluid, fraction, cell, tissue, or subject.

In some embodiments of any of the above aspects, the macromolecule or one or both members of pair of macromolecules comprises a leader sequence. In some embodiments, the leader sequence comprises a secretion signal.

In some embodiments of any of the above aspects, the macromolecule or one or both members of pair of macromolecules comprises a half-life extension moiety. In some embodiments, the half-life extension moiety is an Fc domain or a fragment thereof, an albumin domain or a fragment thereof, or polyethylene glycol (PEG) or a modified derivative thereof.

In another aspect, provided herein is a nucleic acid encoding any of the macromolecules provided herein.

In another aspect, provided herein is a pair of nucleic acids encoding any of the pairs of macromolecules provided herein.

In some embodiments, the nucleic acid is an RNA or a DNA.

In some embodiments, the nucleic acid is formulated with a delivery platform. In some embodiments, the delivery platform is a lipid-based carrier or a vector delivery system. In some embodiments, the lipid-based carrier is a lipid nanoparticle (LNP). In some embodiments, the vector delivery system comprises (or is derived from) an adenovirus, an anellovirus, an AAV, or a lentivirus.

In another aspect, provided herein is a nucleic acid encoding any of the macromolecules provided herein, wherein the nucleic acid is formulated with a carrier.

In another aspect, provided herein is a pair of nucleic acids encoding any of the pairs of macromolecules provided herein, wherein the pair of nucleic acids is formulated with a carrier.

In some embodiments, the nucleic acid is an RNA or a DNA.

In some embodiments, the carrier is a lipid-based carrier. In some embodiments, the lipid-based carrier is a LNP.

In another aspect, provided herein is a vector comprising any of the nucleic acids provided herein.

In another aspect, provided herein is a pair of vectors comprising any of the pairs of nucleic acids provided herein.

In some embodiments, the vector or pair of vectors is formulated with a carrier.

In another aspect, provided herein is a host cell comprising any of the nucleic acids or pairs of nucleic acids or any of the vectors or pairs of vectors provided herein.

In another aspect, provided herein is a multimer comprising two or more of any of the macromolecules provided herein. In some embodiments, each of the macromolecules comprises the same amino acid sequence.

In another aspect, provided herein is a multimer comprising at least one copy of each of any of the pairs of macromolecules provided herein.

In some embodiments of any of the above aspects, the macromolecule complex, macromolecule, nucleic acid, or pair of nucleic acids is about 90%, 91%, 92%, 93%, 94%, 95%, 96%, 97%, 98%, or 99% pure or more than 99% pure.

In some embodiments of any of the above aspects, the macromolecule complex, macromolecule, nucleic acid, or pair of nucleic acids is manufactured according to the U.S. Food and Drug Administration (FDA)'s Good Manufacturing Practice (GMP), Good Clinical Practice (GCP), and/or Good Laboratory Practice (GLP) standards.

In another aspect, provided herein is a method comprising providing any of the macromolecules or pairs of macromolecules, the nucleic acids or pairs of nucleic acids, or the multimers provided herein access to a cell.

In another aspect, provided herein is a method of modulating the state of a cell, comprising providing any of the macromolecules or pairs of macromolecules, the nucleic acids or pairs of nucleic acids, or the multimers provided herein access to a cell, thereby modulating the state of the cell.

In another aspect, provided herein is a method of inducing a cellular effector function in a cell, comprising providing any of the macromolecules or pairs of macromolecules, the nucleic acids or pairs of nucleic acids, or the multimers provided herein access to a cell, thereby inducing the cellular effector function in the cell.

In some embodiments, the cell is in a subject and the macromolecule, nucleic acid, or multimer is administered in a therapeutically effective amount.

In some embodiments, the subject has, or is suspected of having, a disease or disorder characterized by abnormal levels of the disease signature target, optionally wherein the subject was previously determined to have abnormal levels of the disease signature target.

In another aspect, provided herein is a method of determining the state of a cell, comprising providing the any of the macromolecule or pair of macromolecules comprising a reporter domain provided herein, a multimer thereof, or a nucleic acid or pair of nucleic acids encoding the same access to the cell, and detecting the presence of the reporter domain, thereby determining the state of the cell.

In another aspect, provided herein is a method of inducing a cellular effector function in a cell, the method comprising contacting the cell with a macromolecule comprising a first binding domain linked to a second binding domain, wherein (a) the first binding domain specifically binds a disease signature ligand in a biological sample comprising the cell; and (b) the second binding domain specifically binds an effector ligand in the biological sample and induces the cellular effector function upon binding to the effector ligand; wherein the macromolecule is capable of forming a multimer in the presence of the disease signature ligand, and wherein induction of the effector function by the macromolecule is conditional upon each member of the multimer binding the disease signature ligand.

In another aspect, provided herein is a method of inducing a cellular effector function in a cell, the method comprising contacting the cell with a pair of macromolecules, each independently comprising a first binding domain linked to a second binding domain, wherein (a) the first binding domain of each macromolecule specifically binds a disease signature ligand in a biological sample comprising the cell; (b) the second binding domain of a first member of the pair of macromolecules specifically binds a first effector ligand in the biological sample; and (c) the second binding domain of a second member of the pair of macromolecules specifically binds a second effector ligand in the biological sample; wherein the second binding domain of the first member of the pair of macromolecules and the second binding domain of the second member of the pair of macromolecules induce the cellular effector function upon binding to the first effector ligand and the second effector ligand; wherein the pair of macromolecules is capable of forming a heteromultimer in the presence of the disease signature ligand, and wherein induction of the effector function by the macromolecule is conditional upon each member of the heteromultimer binding the disease signature ligand.

In another aspect, provided herein is a pair of macromolecules, each comprising a first binding domain linked to a second binding domain, wherein (a) the first binding domain of a first member of the pair of macromolecules specifically binds a first moiety of a disease signature ligand in a biological sample; (b) the first binding domain of a second member of the pair of macromolecules specifically binds a second moiety of the disease signature ligand in the biological sample; and (c) the second binding domain of each macromolecule specifically binds an effector ligand in the biological sample and induces a cellular effector function upon binding to the effector ligand; wherein the macromolecule is capable of forming a heteromultimer in the presence of the disease signature ligand, and wherein induction of the effector function by the macromolecule is conditional upon each member of the heteromultimer binding the disease signature ligand.

In another aspect, provided herein is a set of three macromolecules, each comprising a first binding domain linked to a second binding domain, wherein (a) the first binding domain of each macromolecule specifically binds a disease signature ligand in a biological sample; (b) the second binding domain of a first member of the set of macromolecules specifically binds a first effector ligand in the biological sample; (c) the second binding domain of a second member of the set of macromolecules specifically binds a second effector ligand in the biological sample; and (d) the second binding domain of a third member of the set of macromolecules specifically binds a third effector ligand in the biological sample; wherein the second binding domain of the first, second, and third members of the pair of macromolecules induce a cellular effector function upon binding to the first, second, and third effector ligands; wherein the set of macromolecules is capable of forming a heterotrimer in the presence of the disease signature ligand, and wherein induction of the effector function by the macromolecule is conditional upon each member of the heterotrimer binding the disease signature ligand.

In some embodiments, the disease signature ligand is trimeric.

In some embodiments, binding of the second binding domain to the effector ligand is conditional upon each member of the multimer binding the disease signature ligand.

In some embodiments, binding of the second binding domain to the effector ligand is not conditional upon each member of the multimer binding the disease signature ligand.

In another aspect, provided herein is a plurality of macromolecules, each independently comprising a first binding domain linked to a second binding domain, wherein (a) the first binding domain of each macromolecule specifically binds a disease signature ligand in a biological sample; and (b) the second binding domain of each macromolecule specifically binds an effector ligand in the biological sample; wherein the plurality of macromolecules is capable of forming a multimer in the presence of the disease signature ligand, and wherein induction of the effector function by the macromolecule is conditional upon each member of the multimer binding a disease signature ligand.

In some embodiments, the multimer is a homodimer, a homotrimer, or a homotetramer.

In some embodiments, the multimer is a heterodimer, a heterotrimer, or a heterotetramer.

In some embodiments, at least one macromolecule comprises a disease signature ligand that is a protein, a peptide, or a small molecule.

In some embodiments, binding of the second binding domain to the effector ligand is conditional upon each member of the multimer binding the disease signature ligand.

In some embodiments, binding of the second binding domain to the effector ligand is not conditional upon each member of the multimer binding the disease signature ligand.

Other features and advantages of the invention will be apparent from the following Detailed Description and the Claims.

### Definitions

As used herein, the term “macromolecule” refers to a large molecule (e.g., a molecule with a size greater than 1000 Daltons (1 kDa)) comprising one or more polypeptide, oligonucleotide, chemical, lipid, and/or carbohydrate moieties. In some embodiments, the macromolecule is a recombinant protein (e.g., a fusion protein).

As used herein, the term “multimer” refers to a molecule made up of at least two subunits (e.g., at least two subunits comprising one or more polypeptide, oligonucleotide, chemical, lipid, and/or carbohydrate moieties). Multimers include homomultimers and heteromultimers. A “homomultimer” is a multimer consisting of two or more identical or substantially identical subunits (e.g., two or more macromolecules). Homomultimers include homodimers (comprising two identical or substantially identical subunits), homotrimers (comprising three identical or substantially identical subunits), and homotetramers (comprising four identical or substantially identical subunits). “Substantially identical subunits” include subunits having differences in amino acid sequences that do not significantly affect the function of the subunit, e.g., that do not significantly affect the affinity of the subunit for one or more ligands. A “heteromultimer” is a multimer consisting of two or more non-identical subunits. Heteromultimers include heterodimers (comprising a first and a second subunit, e.g., comprising a pair of non-identical macromolecules), heterotrimers (comprising one copy of a first subunit and two copies of a second subunit), and homotetramers (comprising two copies or versions of each of a first and a second subunit). Multimers further include higher-order multimers, e.g., hexamers, heptamers, octamers, nonamers, and decamers.

As used herein, the term “binding domain” refers to any domain that has specific affinity for a ligand. Binding domains include, without limitation, polypeptides (e.g., an antibody or a fragment thereof (e.g., an scFv, a monospecific tandem scFv (taFv), a bispecific taFv, a VHH, a VNAR, a Fab, a monospecific single-chain diabody, a bispecific single-chain diabody, or a dual-affinity re-targeting antibody (DART)), an antibody mimetic (e.g., an affibody, an affilin, an affimer, an affitin, an alphabody, an anticalin, a lipocalin, an avimer, a DARPin, a fynomer, a gastrobody, a knottin, a Kunitz domain peptide, a monobody, a fibronectin type III domain (FN3)-based binder, a nanoantibody, a nanoCLAMP, an optimer, a repebody, a pronectin, a centyrin, an obody, a peptide aptamer, a synthetic peptide, or a variable lymphocyte receptor (VLR)), an endogenous binding domain (e.g., a cell receptor domain, an enzyme domain, a variable lymphocyte receptor (VLR) domain, a receptor ectodomain, a nuclear hormone receptor ligand-binding domain, or a DNA-binding domain))), oligonucleotides (e.g., a nucleic acid aptamer (e.g., a DNA aptamer)), and chemical molecules, as well as combinations thereof.

As used herein, the term “ligand” refers to any moiety for which a binding domain as described herein may have affinity. Ligands include, without limitation, a chemical moiety, a portion of a molecule, a molecule (e.g., an allergen or a toxin), a macromolecule (e.g., a polypeptide, a nucleic acid, or carbohydrate), a post-translational modification state of a macromolecule (e.g., a macromolecule that is phosphorylated, glycosylated, acylated, alkylated, and the like), a higher-order macromolecular structure (e.g., a complex of two or more polypeptides), a cell (e.g., a cancer cell), a portion of a cell (e.g., a tumor antigen), a receptor on the surface of a cell, a pathogen (e.g., a virus or a portion or a virus; a bacterium or a portion of a bacterium; a fungus or a portion of a fungus; or a parasite or a portion of a parasite), or a tissue-type.

The term “disease signature ligand” refers to a ligand that is associated with a disease state or a disorder of a cell, tissue, or subject (e.g., mammal, e.g., human). Disease signature ligands may be proteins, e.g., soluble proteins, insoluble proteins, monomeric proteins, and multimeric proteins. Disease signature ligands include, without limitation, cell surface receptors, cell surface antigens, membrane-bound proteins, extracellular matrix components, integrins, cytokines, neurotransmitters, anti-drug antibodies (ADAs), autoantibodies, nucleic acids, carbohydrates, lipids, peptides, nucleosides, hormones, viruses, bacteria, fungi, or a fragment or antigen thereof.

The term “effector ligand” refers to a ligand that is capable of effecting a cellular effector function upon being bound by a binding domain, e.g., a multimer of the invention. Disease signature ligands include proteins and peptides, e.g., cell-surface receptors (e.g., catalytic receptors, e.g., a receptor tyrosine kinase (RTK), a receptor serine/threonine kinase (RSK), a type 1 cytokine receptor, a type 2 cytokine receptor, a tumor necrosis factor (TNF) superfamily receptor (e.g., TNFR2 or 4-1BB), or a nuclear hormone receptor).

As used herein, the term “associated with” a disease, disorder, or condition refers to a relationship, either causative or correlative, between an entity and the occurrence or severity of a disease, disorder, or condition in a subject. For example, if a target is associated with a disease, disorder, or condition, the target may be the causative agent of the disease, disorder, or condition. For example, a virus may be the causative agent in a viral infection, bacteria may be the causative agent in a bacterial infection, a fungus may be the causative agent in a fungal infection, or a parasite may be the causative agent in a parasitic infection, a cancer cell may be the causative agent of a cancer, a toxin may be the causative agent of toxicity, or an allergen may the causative agent of an allergic reaction. The target associated with a disease, disorder, or condition may also or alternately be correlated with an increased likelihood of occurrence or an increased severity of a disease disorder, or condition.

As used herein, the term “carrier” means a compound, composition, reagent, or molecule that facilitates the stability, transport or delivery of a composition (e.g., a macromolecule or pair or macromolecules as described herein) into a subject, a tissue, or a cell. Non-limiting examples of carriers include carbohydrate carriers (e.g., an anhydride-modified phytoglycogen or glycogen-type material), nanoparticles (e.g., a nanoparticle that encapsulates or is covalently linked binds to the circular or linear polyribonucleotide), liposomes, fusosomes, ex vivo differentiated reticulocytes, exosomes, protein carriers (e.g., a protein covalently linked to the polyribonucleotide), and cationic carriers (e.g., a cationic lipopolymer or transfection reagent).

As used herein, the terms “disease,” “disorder,” and “condition” each refer to a state of sub-optimal health, for example, a state that is or would typically be diagnosed or treated by a medical professional.

The term “polynucleotide” as used herein means a molecule comprising one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose. The polynucleotides provided herein may include one or more modified nucleotides.

Polydeoxyribonucleotides or deoxyribonucleic acids, or DNA, means macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, and may include detectable tags, such as luminescent tags or markers (e.g., fluorophores). One or more of the nucleotides may be a modified nucleotide. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular (e.g., a circular RNA). A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may include isolated and purified DNA/RNA molecules, synthetic DNA/RNA molecules, and synthetic DNA/RNA analogs.

As used herein, “polypeptide” means a polymer of amino acid residues (natural or unnatural, including D, L, or a combination thereof) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or may be a multi-molecular complex such as a dimer, trimer, or tetramer. They can also comprise single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the term “sequence identity” is determined by alignment of two peptide or two nucleotide sequences using a global or local alignment algorithm. Sequences may then be referred to as “substantially identical” or “essentially similar” when they (when optimally aligned by for example the programs GAP or BESTFIT using default parameters) share at least a certain minimal percentage of sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively or additionally, percent identity may be determined by searching against databases, using algorithms such as FASTA, BLAST, etc. Sequence identity refers to the sequence identity over the entire length of the sequence.

A “signal sequence” or “leader sequence” refers to a polypeptide sequence, e.g., between 10 and 30 amino acids in length, that is present at the N-terminus of a polypeptide sequence of a nascent protein which targets the polypeptide sequence to the secretory pathway.

As used herein, the term “treat,” or “treating,” refers to a therapeutic treatment of a disease or disorder (e.g., an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, and/or preventing the spread of the disease or disorder as compared to the state and/or the condition of the disease or disorder in the absence of the therapeutic treatment.

### BRIEF DESCRIPTION OF THE DRAWINGS

FIG. 1 is a schematic diagram showing the domain structure of a fusion protein for exerting an effector function on a second target (e.g., a biological effector ligand) conditional on the presence of a first target (e.g., a disease signal ligand). From N- to C-terminus, the fusion protein includes a leader polypeptide; a single-chain variable fragment (scFv) comprising a heavy chain variable domain (VH) and a light chain variable domain (VL) connected by a (G₄S)₃ linker, wherein the VH and VL domains have affinity for the first target; a (G₄S)ₙ linker; VH and VL domains having affinity for the second target after multimerization with a second molecule having the same VH and VL domains, wherein the VH and VL domains are connected by a G₄S linker; and a FLAG affinity tag.

FIG. 2 is a schematic diagram showing the domain structure of a pair of fusion proteins for exerting an effector function on a second target (e.g., a biological effector ligand) conditional on the presence of a first target (e.g., a disease signal ligand). From N- to C-terminus, the fusion proteins include a leader polypeptide; a FLAG affinity tag; a scFv comprising a VH domain and a VL domain connected by a (G₄S)₃ linker, wherein the VH and VL domains have affinity for the first target; a (G₄S)ₙ linker; and a conditional effector domain A (in the first fusion protein) or a conditional effector domain B (in the second fusion protein).

FIG. 3 is a schematic diagram showing the domain structure of a pair of fusion proteins for exerting an effector function on a second target (e.g., a biological effector ligand) conditional on the presence of a first target (e.g., a disease signal ligand). From N- to C-terminus, the fusion protein includes a leader polypeptide; a scFv comprising a VH domain and a VL domain connected by a (G₄S)₃ linker, wherein the VH and VL domains have affinity for the first target; a (G₄S)ₙ linker; a VHH having affinity for a first moiety of the second target (in the first fusion protein (“anti-a”)) or a VHH having affinity for a second moiety of the second target (in the second fusion protein (“anti-b”)); and a FLAG affinity tag.

FIG. 4 is a schematic diagram showing the domain structure of a pair of fusion proteins for exerting an effector function on a second target (e.g., a biological effector ligand) conditional on the presence of a first target (e.g., a disease signal ligand). From N- to C-terminus, the fusion proteins include a leader polypeptide; a VH domain (in the first fusion protein) or a VL domain (in the second fusion protein) of an scFv, wherein the VH and VL domains of the scFv have affinity for the second target when assembled as a functional scFv, wherein the VH and VL domains have low affinity for one another; a GS(G₄S)₃ linker; a scFv comprising a VH domain and a VL domain connected by a (G₄S)₃ linker, wherein the VH and VL domains have affinity for the first target; and a FLAG affinity tag.

FIG. 5A is a schematic diagram showing the domain structure of a fusion protein that modulates the TPO receptor (TpoR) conditional on the presence of TGF-beta1 (TGFb). From N- to C-terminus, the fusion protein includes a mouse immunoglobulin kappa variable 3 (Ms IgKVIII) leader polypeptide; an anti-TGF-beta1 scFv comprising a VH and a VL connected by a (G₄S)₃ linker; a (G₄S), linker; an anti-TpoR scFv comprising a VH and a VL connected by a (G₄S)₃ linker; and a FLAG affinity tag.

FIG. 5B is a schematic diagram showing two copies of the fusion protein of FIG. 5A bound to TGFb and TpoR. The membrane orientation and downstream signaling partners of TpoR are shown.

FIG. 5C is a graph showing secreted alkaline phosphatase (SEAP) activity (gray) in the culture supernatant of TpoR SEAP reporter cells that were stimulated overnight with a constant amount of the fusion protein of FIG. 5A and varying concentrations of TGFb. TPO native cytokine is shown as a positive control for constitutive activity (black).

FIG. 6A is a schematic diagram showing the domain structure of a fusion protein that modulates TpoR conditional on the presence of interleukin-8 (IL-8). From N- to C-terminus, the fusion protein includes a Ms IgKVIII leader polypeptide; an anti-IL-8 scFv comprising a VH and a VL connected by a (G₄S)₃ linker; a (G₄S)ₙ linker; an anti-TpoR scFv comprising a VH and a VL connected by a (G₄S)₃ linker; and a FLAG affinity tag.

FIG. 6B is a schematic diagram showing two copies of the fusion protein of FIG. 6A bound to IL-8 and TpoR. The membrane orientation and downstream signaling partners of TpoR are shown.

FIG. 6C is a graph showing SEAP activity (gray) in the culture supernatant of TpoR SEAP reporter cells that were stimulated overnight with a constant amount of the fusion protein of FIG. 6A and varying concentrations of IL-8. TPO native cytokine is shown as a positive control for constitutive activity (black).

FIG. 7A is a schematic diagram showing the domain structure of a pair of complementary fusion proteins (fusion proteins (a) and (b)) that modulate human interleukin-2 receptor (IL-2R) activity conditional on the presence of TGFb. Fusion protein (a) includes, from N- to C-terminus, a Ms IgKVIII leader peptide; an anti-TGF-beta1 scFv comprising a VH and a VL connected by a (G₄S)₃ linker; a (G₄S)ₙ linker; and a conditional IL-2R effector domain A (IL-2 (N-term)). Fusion protein (b) includes, from N- to C-terminus, a Ms IgKVIII leader peptide; an anti-TGF-beta1 scFv comprising a VH and a VL connected by a (G₄S)₃ linker; a (G₄S)ₙ linker; and a conditional IL-2R effector domain B (IL-2 (C-term)).

FIG. 7B is a schematic diagram showing fusion proteins (a) and (b) of FIG. 7A bound to the IL-2R receptor components IL2Rβ and IL2RγC, respectively, in the absence (left) or presence (right) of TGFb. The membrane orientation and downstream signaling partners of IL-2R are shown.

FIG. 7C is a graph showing SEAP activity in the culture supernatant of IL-2 SEAP reporter cells that were stimulated overnight with a constant amount of fusion proteins (a) and (b) of FIG. 7A and varying concentrations of TGFb (gray squares). IL-2 cytokine is shown as a positive control for constitutive activity (black circles). TGFβ cytokine is shown as a negative control for conditional activity (gray triangles).

FIG. 8A is a schematic diagram showing the domain structure of a pair of fusion proteins for exerting an effector function on a second target (e.g., a biological effector ligand) conditional on the presence of a first target (e.g., a disease signal ligand), in which from N- to C-terminus, the fusion protein includes a leader polypeptide; a scFv comprising a VH domain and a VL domain connected by a (G₄S)₃ linker, wherein the VH and VL domains have affinity for the first target; a (G₄S)ₙ linker; a VHH having affinity for a first moiety of the second target (in the first fusion protein (“anti-a”)) or a VHH having affinity for a second moiety of the second target (in the second fusion protein (“anti-b”)); and a FLAG affinity tag (left); and a set of schematic diagrams showing such a pair of fusion proteins in which the first target is IFN-gamma (IFNg) and the second target is the IL-10 receptor (IL-10R) (right).

FIG. 8B is a schematic diagram showing the pair of fusion proteins of FIG. 8A bound to IFNg and to the IL-10 receptor components IL-10Ra and IL-10Rb. The membrane orientation and downstream signaling partners of IL-10R are shown.

FIG. 8C is a graph showing SEAP activity in the culture supernatant of IL-10 SEAP reporter cells that were stimulated overnight with 10 nM of the pair of fusion proteins of FIG. 8A and varying concentrations of IFNg (gray squares). IL-10 cytokine is shown as a positive control for constitutive activity (black circles). IFNg cytokine is shown as a negative control for conditional activity (gray triangles).

FIG. 9A is a schematic diagram showing the domain structure of a pair of complementary fusion proteins that modulate IL-10R activity conditional on the presence of IL-6. The first fusion protein includes, from N- to C-terminus, a mouse IgKVIII leader peptide; an anti-IL10Ra VHH antibody; a (G₄S)₂ linker; a first anti-IL-6 VHH antibody; a (G₄S)ₙ linker; and a polyhistidine tag. The second fusion protein includes, from N- to C-terminus, a mouse IgKVIII leader peptide; an anti-IL10Rb VHH antibody; a (G₄S)₂ linker; a second anti-IL-6 VHH antibody; a (G₄S)ₙ linker; and a polyhistidine tag.

FIG. 9B is a schematic diagram showing the pair of fusion proteins of FIG. 9A bound to IL-6 and to the IL-10 receptor components IL-10Ra and IL-10Rb. The membrane orientation and downstream signaling partners of IL-10R are shown.

FIG. 9C is a graph showing SEAP activity in the culture supernatant of IL-10 SEAP reporter cells that were stimulated overnight with a constant amount of the pair of fusion proteins of FIG. 9A and varying concentrations of IL-6 (gray circles). IL-10 cytokine is shown as a positive control for constitutive activity (black squares). IL-6 cytokine is shown as a negative control for conditional activity (gray triangles).

FIG. 10A is a schematic diagram showing the domain structure of a pair of complementary fusion proteins that modulate cluster of differentiation 3 (CD3) activity conditional on the presence of IL-8. The first fusion protein includes, from N- to C-terminus, a mouse IgKVIII leader peptide; an anti-CD3 VH domain of an scFv; a GS(G₄S) linker; an anti-IL-8 scFv; and a FLAG affinity tag. The second fusion protein includes, from N- to C-terminus, a mouse IgKVIII leader peptide; an anti-CD3 VL domain of an scFv; a GS(G₄S) linker; an anti-IL-8 scFv; and a FLAG affinity tag.

FIG. 10B is a schematic diagram showing the pair of fusion proteins of FIG. 10A bound to IL-8 and CD3. The membrane orientation and downstream signaling partners of CD3 are shown.

FIG. 10C is a bar graph showing luciferase activity (via NFAT-luc reporter assay) in the culture supernatant of NFAT-luc Jurkat reporter cells that were stimulated for 24 hours with a constant amount of the pair of fusion proteins of FIG. 10A in the presence or absence of 25 nM IL-8. The full-length parental anti-CD3 scFv is shown as a positive control for constitutive activity.

### DETAILED DESCRIPTION

Featured herein are macromolecules that conditionally induce a cellular effector function (e.g., a biological or therapeutic activity) based on the presence of a disease signature ligand, multimers thereof, compositions comprising the same, and methods of using the same.

### I. Compositions

### A. Macromolecules

i. Macromolecules Forming Homodimers

In one aspect, provided herein is a macromolecule comprising a first binding domain linked to a second binding domain, wherein (a) the first binding domain specifically binds a disease signature ligand in a biological sample; and (b) the second binding domain specifically binds an effector ligand in the biological sample and induces a cellular effector function upon binding to the effector ligand; wherein the macromolecule is capable of forming a multimer in the presence of the disease signature ligand (e.g., only forms a multimer in the presence of the disease signature ligand; substantially only forms a multimer in the presence of the disease signature ligand; or preferentially forms a multimer in the presence of the disease signature ligand), and wherein induction of the effector function by the macromolecule is conditional upon each member of the multimer binding the disease signature ligand. In the presence of the disease signature ligand, the multimer may be formed by direct or indirect interaction between the component macromolecules (e.g., may be formed by direct association between the macromolecules or by indirect association (e.g., via the disease signature ligand)). Exemplary first binding domains and disease signature ligands are provided in Section IB herein. Exemplary second binding domains and effector ligands are provided in Section IC herein.

In some embodiments, the multimer is a dimer (i.e., comprises two copies or versions of the macromolecule), a trimer (i.e., comprises three copies or versions of the macromolecule), or a tetramer (i.e., comprises four copies or versions of the macromolecule). Higher-order multimers (e.g., hexamers, heptamers, octamers, nonamers, and decamers) are also contemplated by the invention.

In some embodiments, the multimer is a homomultimer, i.e., a multimer in which each of the two or more macromolecules are identical (e.g., are identical in amino acid sequence and/or nucleotide sequence). For example, in some embodiments, the macromolecule is a polypeptide, and the two or more macromolecules are identical in amino acid sequence. Alternatively, the multimer may comprise two or more versions of the macromolecule that are non-identical in sequence, but that comprise substantially the same first binding domain and second binding domain. For example, the two or more versions of the macromolecule may comprise binding domains that are identical in sequence (e.g., amino acid sequence and/or nucleotide sequence) or that differ in sequence but have substantially the same affinity for the disease signature ligand or the effector ligand.

In some embodiments of the invention, the multimer is a dimer, e.g., a homodimer or a heterodimer.

In some embodiments of the invention, the second binding domain is an antibody or antibody fragment that does not bind the effector ligand when the macromolecule is in a monomeric form. For example, in some embodiments, the second binding domain comprises heavy chain variable domains (VH domains) and light chain variable domains (VL domains) having affinity for the second target, wherein the VH and VL domains are connected by a short linker (e.g., G₄S linker) that does not allow intra-chain pairing of the VH and VL domains. In some embodiments, the second binding domain is a diabody.

In one exemplary embodiment, the macromolecule is a fusion protein comprising, from N- to C-terminus, a leader polypeptide; a first binding domain comprising a single-chain variable fragment (scFv) comprising a heavy chain variable domain (VH) and a light chain variable domain (VL) connected by a (G₄S)₃ linker, wherein the VH and VL domains have affinity for the disease signal ligand; a (G₄S)ₙ linker; a second binding domain comprising VH and VL domains having affinity for the effector ligand, wherein the VH and VL domains are connected by a G₄S linker; and a FLAG affinity tag. In one embodiment, the disease signal ligand is TGF-beta1 and the effector ligand is the TPO receptor.

In another aspect of the invention, provided herein is a multimer (e.g., a dimer, trimer, or tetramer) comprising two or more copies or versions of the macromolecule of the above aspect.

In another aspect of the invention, provided herein is a complex comprising a multimer as described herein in complex with one or both of the disease signature ligand and the effector ligand.

In some embodiments, the macromolecule is a polypeptide. In other embodiments, the macromolecule comprises one or more non-polypeptide components, e.g., comprises one or more nucleic acid or chemical components, as further described below.

In some embodiments, the macromolecule further comprises one or more of a leader domain (e.g., a leader polypeptide), one or more linker domains, and one or more reporter domains, as further described in Section 1(E) below.

### Nucleic Acids, Vectors, and Host Cells

In another aspect of the invention, provided herein is a nucleic acid (e.g., an RNA molecule or a DNA molecule) encoding one or more of any of the macromolecules described above. The nucleic acid may be formulated with a carrier and/or a delivery platform, e.g., a lipid-based carrier (e.g., a lipid nanoparticle (LNP)) and/or a vector delivery system (e.g., an adenovirus, an adeno-associated virus (AAV), an anellovirus, or a lentivirus). Further examples of lipid-based carriers that may be used in the invention are provided in Section I(I) herein. For example, in some aspects, provided herein is a nucleic acid (e.g., an RNA molecule or a DNA molecule, e.g., a circular or linear: RNA molecule or DNA molecule) encoding one or more of any of the macromolecules described above, wherein the nucleic acid is formulated with a carrier, e.g., a lipid carrier, e.g., a LNP. In some aspects, the nucleic acids include one or more modified nucleotides.

Further provided herein are vectors (e.g., plasmids or viral vectors) comprising or encoding any of the above-described nucleic acids. The vector may be formulated with a carrier, e.g., a carrier appropriate for delivery to a target cell (e.g., a mammalian cell), such as, for example, a lipid-containing carrier, such as an LNP-containing formulation.

Further provided herein are host cells that have been modified to comprise the above-described nucleic acids or vectors. Suitable host cells include bacterial and eukaryotic cells (e.g., mammalian cells). In some embodiments, a nucleic acid or vector as described herein is manufactured in and isolated from a host cell.

ii. Macromolecules Forming Heterodimers

In another aspect, provided herein is a pair of macromolecules, each independently comprising a first binding domain linked to a second binding domain, wherein (a) the first binding domain of each macromolecule specifically binds a disease signature ligand in a biological sample; (b) the second binding domain of a first member of the pair of macromolecules specifically binds a first effector ligand in the biological sample; and (c) the second binding domain of a second member of the pair of macromolecules specifically binds a second effector ligand in the biological sample; wherein the second binding domain of the first member of the pair of macromolecules and the second binding domain of the second member of the pair of macromolecules induce a cellular effector function upon binding to the first effector ligand and the second effector ligand; wherein the pair of macromolecules is capable of forming a heteromultimer in the presence of the disease signature ligand (e.g., only form a heteromultimer in the presence of the disease signature ligand; substantially only form a heteromultimer in the presence of the disease signature ligand; or preferentially form a heteromultimer in the presence of the disease signature ligand), and wherein induction of the effector function by the macromolecule is conditional upon each member of the heteromultimer binding the disease signature ligand. The multimer may be formed by direct or indirect interaction between the component macromolecules (e.g., may be formed by direct association between the macromolecules or by indirect association (e.g., via the disease signature ligand)). Exemplary first binding domains and disease signature ligands are provided in Section IB herein. Exemplary second binding domains and effector ligands are provided in Section IC herein.

In some embodiments, the heteromultimer is a dimer (i.e., comprises one copy of each member of the pair of macromolecules).

In another aspect of the invention, provided herein is a heteromultimer (e.g., a dimer) comprising at least one copy of each of the pair of macromolecules of the above aspect.

In another aspect of the invention, provided herein is a complex comprising a heteromultimer as described herein in complex with one, two, or all three of the disease signature ligand, the first effector ligand, and the second effector ligand.

In some embodiments, each member of the pair of macromolecules is a polypeptide. In other embodiments, one or both members of the pair of macromolecules comprises one or more non-polypeptide components, e.g., comprises one or more nucleic acid or chemical components, as further described below.

In another aspect, provided herein is a pair of macromolecules, each comprising a first binding domain linked to a second binding domain, wherein (a) the first binding domain of a first member of the pair of macromolecules specifically binds a first moiety of a disease signature ligand in a biological sample; (b) the first binding domain of a second member of the pair of macromolecules specifically binds a second moiety of the disease signature ligand in the biological sample; and (c) the second binding domain of each macromolecule specifically binds an effector ligand in the biological sample and induces a cellular effector function upon binding to the effector ligand; wherein the macromolecule is capable of forming a heteromultimer in the presence of the disease signature ligand, and wherein induction of the effector function by the macromolecule is conditional upon each member of the heteromultimer binding the disease signature ligand.

In another aspect of the invention, provided herein is a set of three macromolecules, each comprising a first binding domain linked to a second binding domain, wherein (a) the first binding domain of each macromolecule specifically binds a disease signature ligand in a biological sample; (b) the second binding domain of a first member of the set of macromolecules specifically binds a first effector ligand in the biological sample; (c) the second binding domain of a second member of the set of macromolecules specifically binds a second effector ligand in the biological sample; and (d) the second binding domain of a third member of the set of macromolecules specifically binds a third effector ligand in the biological sample; wherein the second binding domain of the first, second, and third members of the pair of macromolecules induce a cellular effector function upon binding to the first, second, and third effector ligands; wherein the set of macromolecules is capable of forming a heterotrimer in the presence of the disease signature ligand, and wherein induction of the effector function by the macromolecule is conditional upon each member of the heterotrimer binding the disease signature ligand. In some embodiments, the disease signature ligand is trimeric.

In some embodiments, one or both members of the pair of macromolecules, or one, two, or all three of the set of three macromolecules, further comprise one or more of a leader domain (e.g., a leader polypeptide), one or more linker domains, and one or more reporter domains, as further described in Section 1(E) below.

### Nucleic Acids, Vectors, and Host Cells

In another aspect of the invention, provided herein is a pair of nucleic acids (e.g., a pair of RNA molecules or a pair of DNA molecules, e.g., a pair of circular or linear RNA molecules or DNA molecules) encoding any of the pairs of macromolecules described above. The pair of nucleic acids may be formulated together or separately with one or more carriers and/or delivery platforms, e.g., a lipid-based carrier (e.g., a lipid nanoparticle (LNP)) or a vector delivery system (e.g., an adenovirus, an AAV, an anellovirus, or a lentivirus). Further examples of lipid-based carriers that may be used in the invention are provided in Section I(I) herein. For example, in some aspects, provided herein is a pair of nucleic acids (e.g., a pair of RNA molecules or a pair of DNA molecules) encoding any of the macromolecules described above, wherein the pair of nucleic acids is formulated with a carrier, e.g., a lipid carrier, e.g., a LNP.

Further provided herein are vectors (e.g., plasmids or viral vectors) comprising or encoding any of the above-described nucleic acids. In some embodiments, each member of the pair of nucleic acids is comprised or encoded by a separate vector. The pair of vectors may be formulated together or separately with a carrier, e.g., a carrier appropriate for delivery to a target cell (e.g., a mammalian cell).

Further provided herein are host cells that have been modified to comprise the above-described nucleic acids or vectors. Suitable host cells include bacterial and eukaryotic cells (e.g., mammalian cells). In some embodiments, a single host cell comprises both members of the pair of nucleic acids or comprises a pair of vectors encoding each member of the pair of nucleic acids. In other embodiments, a first host cell comprises a first member of the pair of nucleic acids (or a vector comprising or encoding the same), and a second host cell comprises a second member of the pair of nucleic acids (or a vector comprising or encoding the same). In some embodiments, a nucleic acid or vector as described herein is manufactured in and isolated from a host cell.

### B. Disease Signature Ligands and First Binding Domains

i. Disease Signature Ligands

The disease signature ligand bound by the macromolecule or pair of macromolecules may be any moiety (e.g., protein, peptide, or small molecule) associated with a disease state or a disorder of a cell, tissue, or subject (e.g., mammal, e.g., human).

In some embodiments, the disease signature ligand is a protein. In some embodiments, the protein is a soluble protein or an insoluble protein. For example, the disease signature ligand may be present in solution in the biological sample (e.g., may be present in the extracellular space) or may be embedded in a membrane present in the biological sample (e.g., may be embedded in a cell membrane).

In some embodiments, the disease signature ligand is a cell surface receptor (e.g., HER2), a cell surface antigen (e.g., prostate specific membrane antigen (PSMA)), a membrane-bound protein (e.g., ASCT2), an extracellular matrix component (e.g., fibronectin or collagen), or an integrin.

In some embodiments, the disease signature ligand is a multimeric protein (e.g., a homomultimeric protein), e.g., a dimeric, trimeric, or tetrameric protein (e.g., an immunologically active multimeric protein). For example, in some embodiments comprising a single species of macromolecule capable of forming a dimer in the presence of the disease signature ligand (e.g., embodiments as described in Section IA(i) herein), the disease signature ligand may be a dimer, such that each macromolecule binds one member of the dimerized disease signature ligand.

In other embodiments, the disease signature ligand is a monomeric protein.

In some embodiments, the disease signature ligand is a cytokine. In some embodiments, the cytokine is an interleukin (e.g., IL-1, IL-2, IL-4, IL-6, IL-8, IL-10, IL-12, IL-15, IL-17, or IL-23); an interferon (e.g., IFN-gamma); a growth factor (e.g., transforming growth factor beta (TGF-beta), granulocyte colony-stimulating factor (GCSF), granulocyte-macrophage colony-stimulating factor (GCSF), epidermal growth factor (EGF), or erythropoietin (EPO)); a chemokine (e.g., monocyte chemoattractant protein-1 (MCP-1) or interferon gamma-induced protein 10 (IP-10; also called CXCL10); or a member of the TNF family (e.g., TNF-alpha). In some aspects, the cytokine is multimerized (e.g., dimerized, trimerized, or tetramerized). In some embodiments, the disease signature ligand is a neurotransmitter (e.g., serotonin, dopamine, or histamine).

In some embodiments, the disease signature ligand is a self antigen of an organism from which the biological sample is derived (e.g., a self antigen produced by a mammalian subject, e.g., a human subject). In some embodiments, the self antigen is an anti-drug antibody (ADA), e.g., an ADA that targets a therapeutic agent used for treatment of the disease state or a disorder of the cell, tissue, or subject. In some embodiments, the self antigen is an autoantibody. In some embodiments, the self antigen is a cancer antigen, e.g., a tumor marker.

In some embodiments, the disease signature ligand is a nucleic acid.

In some embodiments, the disease signature ligand is a carbohydrate, a lipid, a peptide, a nucleoside, or a combination of the foregoing.

In some embodiments, the disease signature ligand is a hormone (e.g., a peptide/protein hormone (e.g., insulin, oxytocin, or a growth hormone)), an amino acid derivative (e.g., melatonin or thyroxine), a steroid (e.g., a glucocorticoid), or an eicosanoid (e.g., prostaglandin).

In some embodiments, the disease signature ligand is a non-self antigen, i.e., an antigen of an organism other than the one from which the biological sample is derived. In some embodiments, the disease signature ligand is a virus, a bacterium, a fungus, or a fragment or antigen thereof (e.g., a virus, bacterium, or fungus that causes a disease state or a disorder of the cell, tissue, or subject or a fragment thereof).

ii. Disease Signature Ligand Binding Domains (First Binding Domains)

Each of the macromolecules provided herein comprises a first binding domain that specifically binds to the disease signature ligand (e.g., binds to a disease signature ligand as described in Section IB (i), above). In some embodiments, the first binding domain permits binding of an additional binding domain to the disease signature ligand (e.g., is designed or selected such that at least two copies of the first binding domain can bind to the disease signature ligand and/or such that the first binding domain of each member of a pair of macromolecules can bind to the disease signature ligand).

In some embodiments, the first binding domain comprises a polypeptide that specifically binds the disease signature ligand.

In some embodiments, the polypeptide is an antibody or a fragment thereof. In some embodiments, the antibody or fragment thereof is an scFv, a monospecific tandem scFv (taFv), a bispecific taFv, a VHH, a VNAR, a Fab, a monospecific single-chain diabody, a bispecific single-chain diabody, or a dual-affinity re-targeting antibody (DART).

In some embodiments, the polypeptide is an antibody mimetic. In some embodiments, the antibody mimetic is an affibody, an affilin, an affimer, an affitin, an alphabody, an anticalin, a lipocalin, an avimer, a DARPin, a fynomer, a gastrobody, a knottin, a Kunitz domain peptide, a monobody, a fibronectin type III domain (FN3)-based binder, a nanoantibody, a nanoCLAMP, an optimer, a repebody, a pronectin, a centyrin, an obody, a peptide aptamer, a synthetic peptide, or a variable lymphocyte receptor (VLR).

In some embodiments, the polypeptide is an endogenous binding domain of an organism from which the biological sample and/or the disease signature ligand is derived, e.g., a binding domain that is naturally produced by the organism. In some embodiments, the endogenous binding domain is a cell receptor domain, an enzyme domain, a variable lymphocyte receptor (VLR) domain, a receptor ectodomain, a nuclear hormone receptor ligand-binding domain, or a DNA-binding domain. For example, in some embodiments, the first binding domain is a polypeptide that comprises or consists of a receptor for a cytokine (e.g., a multimerized cytokine, e.g., a dimeric, trimeric, or tetrameric cytokine) or is a polypeptide that comprises or consists of a receptor for an immunologically active multimer (e.g., an immunologically active dimer, trimer, or tetramer). In some embodiments, the disease signature ligand is IL-6 and the first binding domain is a polypeptide comprising the IL-6 receptor. In some embodiments, the disease signature ligand is TNF-alpha and the first binding domain is a polypeptide comprising the TNF-alpha receptor.

In some embodiments, the first binding domain comprises an oligonucleotide that specifically binds the disease signature ligand. In some embodiments, the oligonucleotide is a nucleic acid aptamer (e.g., a DNA aptamer).

In some embodiments, the first binding domain comprises a chemical molecule that specifically binds the disease signature ligand.

Additional binding domains that may be used in the invention are described, e.g., in Zhong and D'Antona, Antibodies, 10 (2): 13, 2021.

In some embodiments, the disease signature ligand-binding domain has an affinity (K_{D} value) for the disease signature ligand of >10 to >100 pM, <10 nM, >10 nM, or >100 nM or has micromolar affinity for the disease signature ligand (e.g., K_{D} of ≤1 μM). In some aspects, the disease signature ligand-binding domain binds the disease signature ligand with a Ko of 1 nM or lower.

In some embodiments of any of the macromolecules provided herein, the first binding domain has affinity to two or more disease signature moieties. For example, the first binding domain may comprise at least two binding moieties as described above, wherein the at least two binding moieties specifically bind to at least two different disease signature moieties.

In some embodiments comprising pairs of macromolecules (e.g., embodiments as described in Sections IA(i) and IA(ii) herein), the first and second members of the pair of macromolecules comprise first binding domains that are identical in sequence (e.g., amino acid sequence and/or nucleotide sequence) or that differ in sequence, but have substantially the same affinity for the disease signature ligand or the effector ligand. Alternatively, in other embodiments, the first and second members of the pair of macromolecules comprise different first binding domains. For example, in some embodiments, the first binding domain of a first member of the pair of macromolecules specifically binds a first epitope or moiety of a disease signature ligand in a biological sample, and the second binding domain of a second member of the pair of macromolecules specifically binds a second epitope or moiety of the disease signature ligand in the biological sample.

For example, in some embodiments, the disclosure provides a pair of macromolecules, each independently comprising a first binding domain linked to a second binding domain, wherein (a) the first member of the pair of macromolecules comprises a first binding domain 1 (FBD1) that specifically binds a first epitope of a disease signature ligand in a biological sample; (b) the second member of the pair of macromolecules comprises a first binding domain 2 (FBD2) that specifically binds a second epitope of the disease signature ligand in a biological sample; (c) the second binding domain of the first member of the pair of macromolecules specifically binds a first effector ligand in the biological sample; and (d) the second binding domain of the second member of the pair of macromolecules specifically binds a second effector ligand in the biological sample; wherein the second binding domain of the first member of the pair of macromolecules and the second binding domain of the second member of the pair of macromolecules induce a cellular effector function upon binding to the first effector ligand and the second effector ligand; wherein the pair of macromolecules is capable of forming a heteromultimer in the presence of the disease signature ligand, and wherein induction of the effector function by the macromolecule is conditional upon each member of the heteromultimer binding the disease signature ligand.

In a further example, in some embodiments, the disclosure provides a pair of macromolecules, each independently comprising a first binding domain linked to a second binding domain, wherein (a) the first member of the pair of macromolecules comprises a first binding domain 1 (FBD1) that specifically binds a first epitope of a disease signature ligand in a biological sample; (b) the second member of the pair of macromolecules comprises a first binding domain 2 (FBD2) that specifically binds a second epitope of the disease signature ligand in a biological sample; and (c) the second binding domain specifically binds an effector ligand in the biological sample and induces a cellular effector function upon binding to the effector ligand; wherein the macromolecule is capable of forming a multimer in the presence of the disease signature ligand, and wherein induction of the effector function by the macromolecule is conditional upon each member of the multimer binding the disease signature ligand. In some embodiments, the pair of macromolecules each comprise second binding domains that are identical in sequence (e.g., amino acid sequence and/or nucleotide sequence) or that differ in sequence, but have substantially the same affinity for the effector ligand.

In embodiments comprising non-identical first binding domains (e.g., a first binding domain 1 (FBD1) and a first binding domain 2 (FBD2)), the first binding domains may bind to different epitopes of the disease signature ligand, e.g., partially overlapping epitopes or non-overlapping epitopes, e.g., such that the two first binding domains do not sterically hinder each other from binding their respective epitopes on the disease signature ligand; only partially sterically hinder each other from binding their respective epitopes on the disease signature ligand; or do not completely hinder each other from binding their respective epitopes on the disease signature ligand. Accordingly, in some aspects, the first binding domains are able to concurrently bind the disease signature ligand.

In some embodiments comprising non-identical first binding domains (e.g., an FBD1 and an FBD2), the two first binding domains have substantially similar affinities for their respective epitopes (e.g., affinities that differ by less than 5%, 6%, 7%, 8%, 9%, 10%, 11%, 12%, 13%, 14%, 15%, 16%, 17%, 18%, 19%, 20%, 25%, 30%, 35%, 40%, 45%, or 50%). In other embodiments, the FBD1 and FBD2 have substantially different affinities for their respective epitopes (e.g., affinities that differ by at least 50%, 55%, 60%, 65%, 70%, 75%, 80%, 85%, 90%, 95%, 100%, or more, e.g., 200%, 300%, 400%, 500%, 600%, 700%, 800%, 900%, 1000%, 2500%, 5000%, 7500%, or 10000%).

In some embodiments in which the disease signature ligand is a polypeptide, the first binding domain binds to the disease signature ligand at a binding site that contains an Arg, Lys, Asp, His or Glu amino acid residue (or a combination thereof) (e.g., a binding site that is enriched for one or more of these residues).

In one embodiment, the disease signature ligand is IL-10 and the first binding domain is a polypeptide comprising the IL-10 receptor.

In one embodiment, the disease signature ligand is IL-12 and the first binding domain comprises the p40 subunit and/or the p35 subunit of IL-12.

### C. Effector Ligands and Second Binding Domains

i. Effector Ligands

The effector ligand bound by the macromolecule or pair of macromolecules may be any moiety (e.g., protein or peptide) that is present in the biological sample and is capable of effecting a cellular effector function upon being bound by a multimer of the invention.

In some embodiments, the effector ligand is a protein or a peptide.

In some embodiments, the effector ligand is a cell-surface receptor.

In some embodiments, the cell-surface receptor is a catalytic receptor, e.g., a receptor tyrosine kinase (RTK), a receptor serine/threonine kinase (RSK), a type 1 cytokine receptor, a type 2 cytokine receptor, or a tumor necrosis factor (TNF) superfamily receptor (e.g., TNFR2 or 4-1BB).

In some embodiments, the RTK is VEGFR, the RSK is TGFBR2, the type 1 cytokine receptor is IL2R, the type 2 cytokine receptor is IL10R, or the TNF superfamily receptor is TNFR2.

In some embodiments, the effector ligand is an intracellular receptor. In some embodiments, the intracellular receptor is a nuclear hormone receptor (e.g., a glucocorticoid receptor). In some embodiments in which the effector ligand is an intracellular receptor, the macromolecule is delivered as an RNA.

ii. Mechanisms of Effector Ligand Activation

In some embodiments, the second binding domain is an agonist of the effector ligand. In other embodiments, the second binding domain is an antagonist of the effector ligand.

In some embodiments, the effector ligand must be homodimerized to exert a cellular effector function. In some embodiments, the effector ligand is capable of homodimerization and exerts a cellular effector function with at least 2-fold, 5-fold, 10-fold, 100-fold, or 1000-fold greater strength in the homodimerized form as compared to a monomeric form. In some embodiments, the effector ligand is homodimerized in the presence of the multimer of the macromolecule. In some embodiments, the effector ligand is activated by homodimerization in the absence of its endogenous ligand.

In some embodiments, the macromolecule or pair of macromolecules exhibits conditional avidity, triggered avidity, and/or dimerization avidity. For example, in some aspects, a plurality of macromolecules or pairs of macromolecules bind to a disease signature ligand (e.g., a tumor antigen or a pathogen surface marker), and the proximity results in increased avidity which then activates a potent downstream effect.

In some embodiments comprising pairs of macromolecules (e.g., embodiments as described in Section IA(ii) herein), the first effector ligand and the second effector ligand must be associated (e.g., in proximity and correctly oriented (e.g., heterodimerized)) to exert a cellular effector function. In some embodiments, the effector ligand is capable of heterodimerization and exerts a cellular effector function with at least 2-fold, 5-fold, 10-fold, 100-fold, or 1000-fold greater strength in the heterodimerized form as compared to a monomeric form. Accordingly, in some embodiments, the first effector ligand and the second effector ligand are associated (e.g., in proximity and correctly oriented (e.g., heterodimerized)) in the presence of the multimer of the macromolecule.

In other embodiments comprising pairs of macromolecules (e.g., embodiments as described in Section IA(ii) herein), the first effector ligand and the second effector ligand must be associated (e.g., in proximity and correctly oriented (e.g., heterodimerized)) and must further be associated with one or more additional moieties to exert a cellular effector function. For example, in some embodiments, the first effector ligand and the second effector ligand are members of a receptor complex comprising at least three members (e.g., a homotrimeric receptor complex, a heterotrimeric receptor complex, a homotetrameric receptor complex, or a heterotetrameric receptor complex).

In some embodiments of any of the macromolecules and pairs of macromolecules provided herein, the disease signature ligand is a soluble protein (e.g., a cytokine) and the effector ligand is a catalytic receptor (e.g., a catalytic receptor that exerts a cellular function upon multimerization (e.g., homomultimerization or heteromultimerization)).

iii. Cellular Effector Functions

In some embodiments, the cellular effector function of the disease signature ligand is a biological activity.

In some embodiments, the cellular effector function of the disease signature ligand is a therapeutic activity.

In some embodiments, the cellular effector function of the disease signature ligand is a disease activity (e.g., an aberrant activity associated with a disease state), and the cellular effector function is repressed by binding of the multimerized macromolecule or pair of macromolecules to the effector ligand.

iv. Effector Ligand Binding Domains (Second Binding Domains)

In some embodiments, the second binding domain comprises a polypeptide that specifically binds the effector ligand. In some embodiments, the first binding domain permits binding of an additional binding domain to the disease signature ligand (e.g., is designed or selected such that at least two copies of the second binding domain can bind to the effector ligand).

In some embodiments, the polypeptide is an antibody or a fragment thereof. In some embodiments, the antibody or fragment thereof is an scFv, a monospecific tandem scFv (taFv), a bispecific taFv, a VHH, a VNAR, a Fab, a monospecific single-chain diabody, a bispecific single-chain diabody, or a dual-affinity re-targeting antibody (DART).

In some embodiments, the polypeptide is an antibody mimetic. In some embodiments, the antibody mimetic is an affibody, an affilin, an affimer, an affitin, an alphabody, an anticalin, a lipocalin, an avimer, a DARPin, a fynomer, a gastrobody, a knottin, a Kunitz domain peptide, a monobody, a fibronectin type III domain (FN3)-based binder, a nanoantibody, a nanoCLAMP, an optimer, a repebody, a pronectin, a centyrin, an obody, a peptide aptamer, a synthetic peptide, or a variable lymphocyte receptor (VLR).

In some embodiments, the polypeptide is an endogenous binding domain. In some embodiments, the endogenous binding domain is a ligand of the effector ligand or a fragment thereof. In some embodiments, the endogenous binding domain is a viral binding protein or a fragment thereof.

In some embodiments, the second binding domain comprises an oligonucleotide that specifically binds the effector ligand. In some embodiments, the oligonucleotide is a nucleic acid aptamer (e.g., a DNA aptamer).

Additional binding domains that may be used in the invention are described, e.g., in Zhong and D'Antona, Antibodies, 10 (2): 13, 2021.

In some embodiments, the effector ligand-binding domain has an affinity for the effector ligand that is similar to that of a native ligand of the effector ligand. In some embodiments, the effector ligand-binding domain has an affinity (K_{D} value) for the effector ligand that is in the picomolar (μM) range or is <1 μM. In some embodiments, the effector ligand-binding domain has an affinity for the effector ligand that is 500 nM or lower. In some embodiments in which the effector ligand binding domain comprises two monomers, the affinity of each of the monomers for the effector ligand is 1-2 orders of magnitude higher than the affinity of the effector ligand binding domain monomers for one another.

In some embodiments of any of the macromolecules provided herein, the second binding domain has affinity to two or more effector ligands. For example, the second binding domain may comprise at least two binding moieties as described above, wherein the at least two binding moieties specifically bind to at least two different effector moieties.

### Pairs of Second Binding Domains

In some embodiments comprising pairs of macromolecules (e.g., embodiments as described in Section IA(ii) herein), the first and second members of the pair of macromolecules comprise second binding domains that are identical in sequence (e.g., amino acid sequence and/or nucleotide sequence) or that differ in sequence, but have substantially the same affinity for the disease signature ligand or the effector ligand. Alternatively, in other embodiments, the first and second members of the pair of macromolecules comprise different second binding domains. For example, in some embodiments, the second binding domain of a first member of the pair of macromolecules specifically binds a first effector ligand in a biological sample, and the second binding domain of a second member of the pair of macromolecules specifically binds a second effector ligand in the biological sample.

For example, in some embodiments comprising pairs of macromolecules, the second binding domain of the first member of the pair of macromolecules is a first portion of a binding moiety and the second binding domain of the second member of the pair of macromolecules is a second portion of the binding moiety.

In one exemplary embodiment, the pair of macromolecules is a pair of polypeptides comprising, from N- to C-terminus, a leader polypeptide; a VH domain (in the first fusion protein) or a VL domain (in the second fusion protein) of an scFv having affinity for the effector ligand, wherein the VH and VL domains have low affinity for one another; a GS(G₄S)₃ linker; a scFv comprising a VH domain and a VL domain connected by a (G₄S)₃ linker, wherein the VH and VL domains have affinity for the disease signal ligand; and a FLAG affinity tag. In some embodiments, the disease signature ligand is TGF-beta1 and the effector ligand is cluster of differentiation 3 (CD3).

In another example, the second binding domain of the first member of the pair of macromolecules specifically binds to a first component of a heteromultimeric (e.g., heterodimeric) receptor and the second binding domain of the second member specifically binds to a second component of the heteromultimeric (e.g., heterodimeric) receptor.

In one exemplary embodiment, the pair of macromolecules is a pair of polypeptides, wherein (i) a first member of the pair of polypeptides comprises, from N- to C-terminus, a leader polypeptide; a scFv comprising a VH domain and a VL domain connected by a (G₄S)₃ linker, wherein the VH and VL domains have affinity for the disease signature ligand; a (G₄S)ₙ linker; a VHH having affinity for a first component of a heteromultimeric (e.g., heterodimeric) receptor; and a FLAG affinity tag, and (ii) a second member of the pair of polypeptides comprises, from N- to C-terminus, a leader polypeptide; a scFv comprising a VH domain and a VL domain connected by a (G₄S)₃ linker, wherein the VH and VL domains have affinity for the disease signature ligand; a (G₄S)ₙ linker; a VHH having affinity for a second component of the heteromultimeric (e.g., heterodimeric) receptor; and a FLAG affinity tag. In one embodiment, the disease signature ligand is IFN-gamma and the VHH of the first and second polypeptides have affinity for IL10Ra and IL10Rb, respectively.

In another example, the second binding domain of the first member of the pair of macromolecules is a first component of a dimeric moiety and the second binding domain of the second member is a second component of a dimeric moiety.

In another example, the second binding domain of the first member of the pair of macromolecules is a first fragment of a polypeptide chain and the second binding domain of the second member is a second fragment of the polypeptide chain. In some embodiments, the polypeptide chain is a hormone, a cytokine, or a growth factor.

In one exemplary embodiment, the pair of macromolecules is a pair of polypeptides, wherein (i) a first member of the pair of polypeptides comprises, from N- to C-terminus, a leader polypeptide; a FLAG affinity tag; a first binding domain consisting of an scFv comprising a VH domain and a VL domain connected by a (G₄S)₃ linker, wherein the VH and VL domains have affinity for the disease signal ligand; a (G₄S)ₙ linker; and a second binding domain comprising a first portion of a binding moiety, and (ii) a second member of the pair of polypeptides comprises a leader polypeptide; a FLAG affinity tag; a first binding domain consisting of an scFv comprising a VH domain and a VL domain connected by a (G₄S)₃ linker, wherein the VH and VL domains have affinity for the first target; a (G₄S)ₙ linker; and a second binding domain comprising a second portion of a binding moiety. In one embodiment, the disease signal ligand is TGF-beta1, the effector ligand is the IL-2 receptor, and the binding moiety is IL-2.

In some embodiments of any of the above examples, the second binding domain of the first member of the pair of macromolecules and the second binding domain of the second member of the pair of macromolecules have been engineered to have reduced affinity for one another. In some embodiments, the second binding domain of the first member of the pair of macromolecules and the second binding domain of the second member of the pair of macromolecules have an affinity (K_{D}) for one another of >1 μM, e.g., >5-10 μM, but less than 1 mM (e.g., 10-200 μM).

In some embodiments of any of the macromolecules or pairs of macromolecules described herein, the second binding domain comprises a conditional effector domain.

### D. Pairs of First and Second Binding Domains

In some embodiments, the disease signature ligand is TGFbeta and the effector ligand is the TPO receptor. For example, in some embodiments, the first binding domain comprises a TGFbeta-binding domain (e.g., an anti-TGF-beta1 antibody or antibody fragment, e.g., an anti-TGF-beta1 single-chain variable fragment (scFv)) and the second binding domain comprises a TPO receptor (TpoR) binding domain (e.g., an anti-TpoR antibody or antibody fragment, e.g., an anti-TpoR heavy chain variable domain (VH) and light chain variable domain (VL)).

In some embodiments, the disease signature ligand is IL-8 and the effector ligand is the TPO receptor. For example, in some embodiments, the first binding domain comprises an IL-8-binding domain (e.g., an anti-IL-8 antibody or antibody fragment, e.g., an anti-IL-8 scFv) and the second binding domain comprises a TpoR binding domain (e.g., an anti-TpoR antibody or antibody fragment, e.g., an anti-TpoR VH and VL).

In some embodiments, the disease signature ligand is TGFbeta and the effector ligand is the IL-2 receptor (IL-2R). For example, in some embodiments, the first binding domain comprises a TGFbeta-binding domain (e.g., an anti-TGF-beta1 antibody or antibody fragment, e.g., an anti-TGF-beta1 scFv) and the one or more second binding domains comprise an IL-2R binding domain (e.g., a fragment and/or modified version of IL-2). In some embodiments, the disclosure provides a macromolecule or pair of macromolecules comprising two second binding domains comprising complementary fragments of IL-2R.

In some embodiments, the disease signature ligand is IL-8 and the effector ligand is the IL-2 receptor (IL-2R). For example, in some embodiments, the first binding domain comprises an IL-8-binding domain (e.g., an anti-IL-8 antibody or antibody fragment) and the one or more second binding domains comprise an IL-2R binding domain (e.g., a fragment and/or modified version of IL-2). In some embodiments, the disclosure provides a macromolecule or pair of macromolecules comprising two second binding domains comprising complementary fragments of IL-2R.

In some embodiments, the disease signature ligand is IFN-gamma and the effector ligand is the IL-10 receptor. For example, in some embodiments, the first binding domain comprises an IFN-gamma-binding domain (e.g., an anti-IFN-gamma antibody or antibody fragment, e.g., an anti-IFN-gamma scFv) and the one or more second binding domains comprise an IL-10-receptor binding domain (e.g., an antibody or antibody fragment targeting one or more components of the IL-10 receptor). In some embodiments, the disclosure provides a macromolecule or pair of macromolecules comprising two second binding domains targeting different components of the IL-10 receptor, e.g., IL-10Ra and IL-10Rb.

In some embodiments, the disease signature ligand is IL-6 and the effector ligand is the IL-10 receptor. For example, in some embodiments, the first binding domain comprises an IL-6-binding domain (e.g., an anti-IL-6 antibody or antibody fragment, e.g., an anti-IL-6 VHH antibody) and the one or more second binding domains comprise an IL-10-receptor binding domain (e.g., an antibody or antibody fragment targeting one or more components of the IL-10 receptor). In some embodiments, the disclosure provides a macromolecule or pair of macromolecules comprising two second binding domains targeting different components of the IL-10 receptor, e.g., IL-10Ra and IL-10Rb and/or two first binding domains targeting different epitopes of IL-6.

In some embodiments, the disease signature ligand is TGFbeta and the effector ligand is cluster of differentiation 3 (CD3). For example, in some embodiments, the first binding domain comprises a TGFbeta-binding domain (e.g., an anti-TGF-beta1 antibody or antibody fragment, e.g., an anti-TGF-beta1 single-chain variable fragment (scFv)) and the one or more second binding domains comprise a CD3-binding domain (e.g., an anti-CD3 antibody or antibody fragment, e.g., an anti-CD3 heavy chain variable domain (VH) and/or light chain variable domain (VL)). In some embodiments, the disclosure provides a macromolecule or pair of macromolecules comprising two second binding domains comprising different components of a CD3-targeting moiety, e.g., respectively comprising an anti-CD3 VH and VL.

In some embodiments, the disease signature ligand is IL-8 and the effector ligand is CD3. For example, in some embodiments, the first binding domain comprises an IL-8-binding domain (e.g., an anti-IL-8 antibody or antibody fragment, e.g., an anti-IL-8 single-chain variable fragment (scFv)) and the one or more second binding domains comprise a CD3-binding domain (e.g., an anti-CD3 antibody or antibody fragment, e.g., an anti-CD3 heavy chain VH and/or VL). In some embodiments, the disclosure provides a macromolecule or pair of macromolecules comprising two second binding domains comprising different components of a CD3-targeting moiety, e.g., respectively comprising an anti-CD3 VH and VL.

In some embodiments, the disease signature ligand-binding domain (first binding domain) is part of an inflammatory cytokine system and the effector ligand-binding domain (second binding domain) comprises IL-10 or a fragment and/or modified version thereof. In some aspects, the effector ligand is the IL-10 receptor.

In some embodiments, the disease signature ligand-binding domain (first binding domain) comprises TNFalpha, MCP-1, or IL-12 or a fragment and/or modified version thereof and the effector ligand-binding domain (second binding domain) comprises IL-10 or a fragment and/or modified version thereof. In some aspects, the effector ligand is the IL-10 receptor.

### E. Leader, Reporter and Linker Moieties

### Leaders

In some embodiments of any of the compositions and methods provided herein, the macromolecule comprises a leader peptide, e.g., a leader peptide that targets the macromolecule for secretion. The leader peptide may be cleaved from the macromolecule prior to formation of the multimer. In some embodiments, the leader peptide is a mouse immunoglobulin kappa variable 3 (IgKVIII) leader peptide (e.g., UniProt ID A0A140T8P0 positions M1 to G20). Further exemplary leader sequences are provided in Table 1 (SEQ ID NOs: 15-31).

TABLE 1
Leader sequences

| Leader sequence name | SEQ ID number | Sequence |
|---|---|---|
| Human OSM | SEQ ID NO: 15 | MGVLLTQRTLLSLVLALLFPSMASM |
| VSV-G | SEQ ID NO: 16 | MKCLLYLAFLFIGVNC |
| Mouse Ig Kappa | SEQ ID NO: 17 | METDTLLLWVLLLWVPGSTGD |
| Mouse Ig Heavy | SEQ ID NO: 18 | MGWSCIILFLVATATGVHS |
| BM40 | SEQ ID NO: 19 | MRAWIFFLLCLAGRALA |
| Secrecon | SEQ ID NO: 20 | MWWRLWWLLLLLLLLWPMVWA |
| Human IgKVIII | SEQ ID NO: 21 | MDMRVPAQLLGLLLLWLRGARC |
| CD33 | SEQ ID NO: 22 | MPLLLLLPLLWAGALA |
| tPA | SEQ ID NO: 23 | MDAMKRGLCCVLLLCGAVFVSPS |
| Human Chymotrypsinogen | SEQ ID NO: 24 | MAFLWLLSCWALLGTTFG |
| Human trypsinogen-2 | SEQ ID NO: 25 | MNLLLILTFVAAAVA |
| Human IL-2 | SEQ ID NO: 26 | MYRMQLLSCIALSLALVTNS |
| Gaussia luc | SEQ ID NO: 27 | MGVKVLFALICIAVAEA |
| Albumin(HSA) | SEQ ID NO: 28 | MKWVTFISLLFSSAYS |
| Influenza Haemagglutinin | SEQ ID NO: 29 | MKTIIALSYIFCLVLG |
| Human insulin | SEQ ID NO: 30 | MALWMRLLPLLALLALWGPDPAAA |
| Silkworm Fibroin LC | SEQ ID NO: 31 | MKPIFLVLLVVTSAYA |

### Reporters

In some embodiments of any of the compositions and methods provided herein, the macromolecule comprises a reporter moiety. For example, in embodiments comprising a pair of macromolecules, one or both members of the pair of macromolecules may comprise the reporter moiety, or the members of the pair of macromolecules may each comprise different reporter moieties.

Exemplary reporter moieties include, without limitation, affinity tags (e.g., FLAG affinity tags), fluorescent markers, and chromogenic markers (e.g., luciferase or beta-lactamase). For example, in some embodiments, the reporter moiety is a near-infrared probe (e.g., indocyanine green (ICG) or methylene blue (MB)) or a near-infrared fluorescent protein or a fragment thereof. In other embodiments, the reporter moiety comprises a fragment of a bait protein and is detected by adding an exogenous dye that detects the bait protein.

In some embodiments comprising a pair of macromolecules, the first and second members of the pair of macromolecules comprise complementary reporter moieties, e.g., reporter moieties that are detectable (e.g., produce a fluorescent signal) when the first and second members of the pair of macromolecules form a multimer. For example, the first and second members of the pair of macromolecules may comprise members of a fluorescence resonance energy transfer (FRET) pair (e.g., a near-infrared FRET pair), e.g., a peptide-based or protein-based FRET pair.

### Linkers

In some embodiments of any of the compositions and methods provided herein, the macromolecule comprises one or more linker domains, e.g., linker domains that connect the first binding domain to the second binding domain; connect one or more sub-domains within the first binding domain or the second binding domain; and/or connect the first binding domain or the second binding domain to a leader peptide or a reporter moiety.

In some embodiments, the one or more linker domains are peptide linkers. In some embodiments, the peptide linkers are GS linkers. In some embodiments, the peptide linkers are glycine-serine (GS) linkers, e.g., GS linkers having the format GS(GₙS)ₘ or GS linkers having the format (GₙS)ₘ, e.g., wherein n=1, 2, 3, 4, 5, 6, 7, 8, 9, or 10 (e.g., n=1-5 or 5-10, e.g., n=4) and m=1, 2, 3, 4, 5, 6, 7, 8, 9, 10, 11, 12, 13, 14, or 15 (e.g., m=1-5, 5-10, or 10-15, e.g., m=5).

### F. Half-Life Extension Moieties

Any of the macromolecules (e.g., polypeptides) provided herein may be modified to alter (e.g., extend) their half-life (e.g., to alter (e.g., extend) their half-life (e.g., half, life in circulation (e.g., in serum)) and/or to elicit a desired effector function. For example, in some embodiments, any of the macromolecules provided herein may include a moiety (e.g., a heterologous moiety) that extends the half-life of the macromolecule. Exemplary half-life extension moieties include polypeptides (e.g., a fragment crystallizable region (Fc region) or a fragment or variant thereof or an albumin domain or a fragment or variant thereof) and non-polypeptide moieties (e.g., polyethylene glycol (PEG) or a modified derivative thereof).

In some aspects, a polypeptide provided herein is modified to include a Fc region that extends the half-life of the polypeptide relative to a version of the polypeptide not comprising the Fc region. In some embodiments, the Fc region is an IgG isotype Fc region, e.g., an IgG1, IgG2, or IgG4 subtype Fc region (e.g., such an Fc region from a human, a mouse, or a non-human primate (NHP)). In some embodiments, the Fc region comprises one or more Fc effector function-silencing mutations (e.g., LALA or LALAPG mutations (mutations in IgG1 at positions L234, L235, G236, N297, or P329)); in other aspects, the Fc region is capable of eliciting one or more Fc effector functions. The Fc region may be modified to extend half-life using one or more mutations that enhance neonatal Fc receptor (FcRn)-based recycling. Further Fc variants that may be used in the invention include mutated Fc variants previously described to alter Fc gamma receptor binding or Fc neonatal receptor binding and recycling and Fc variants comprising glycosylation modifications. Variant Fc regions that may be used in the invention include those provided in Saunders, Frontiers in Immunology, 10: Article 1296, 2019; Delidakis et al., Annual Review of Biomedical Engineering, 24:249-274, 2022; and Wilkinson et al., PLOS ONE, 16 (12): e0260954, 2021.

In some aspects, a polypeptide provided herein is modified to include an Fc region that alters Fc gamma receptor binding and/or effector function or Fc neonatal receptor binding and/or recycling. In some aspects, a polypeptide provided herein is modified to include an Fc region that comprises one or more glycosylation modifications.

In some aspects, a macromolecule (e.g., polypeptide) provided herein is modified to include a human serum albumin (HSA) or binder thereof that extends the half-life (e.g., half-life in circulation) of the polypeptide relative to a version of the polypeptide not comprising the HSA or binder thereof. For example, in some embodiments, the polypeptide is directly fused to HSA. In other embodiments, the polypeptide is fused to a HSA binder, e.g., a short peptide sequence, a VHH, or any other antibody or natural scaffold that targets HSA.

In some aspects, the modification (e.g., heterologous moiety) increases the half-life of the macromolecule (e.g., polypeptide) by at least 1%, 5%, 10%, 15%, 20%, 25%, 30%, 35%, 40%, 45%, 50%, 55%, 60%, 65%, 70%, 75%, 80%, 85%, 90%, 95%, 100%, or more than 100% (e.g., 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or 95-100%) relative to a control macromolecule, e.g., a version of the macromolecule not comprising the modification.

### G. Manufacturing and Purity

In some embodiments of any of the compositions and methods provided herein, the macromolecule, pair of macromolecules, nucleic acid, pair of nucleic acids, multimer, or composition comprising the same is at least 95% pure (e.g., at least 95% free of any impurity or undesired substance). In some embodiments, the macromolecule, pair of macromolecules, macromolecule complex, nucleic acid, pair of nucleic acids, multimer, or composition comprising the same is more than 95% pure, e.g., is at least 96%, 97%, 98%, or 99% pure or is 100% pure.

In some embodiments of any of the compositions and methods provided herein, the macromolecule, pair of macromolecules, nucleic acid, pair of nucleic acids, multimer, or composition comprising the same are manufactured in accordance with one or more International Organization for Standardization (ISO) standards.

In some embodiments, the macromolecule, pair of macromolecules, macromolecule complex, nucleic acid, pair of nucleic acids, multimer, or composition comprising the same is manufactured according to the U.S. Food and Drug Administration (FDA)'s Good Manufacturing Practice (GMP), Good Clinical Practice (GCP), and/or Good Laboratory Practice (GLP) standards.

### H. Biological Samples

In some embodiments of any of the compositions and methods provided herein, the biological sample is an extract, fluid, or fraction (e.g., an extract, fluid, or fraction derived from a subject and comprising a cell); a cell; a tissue; or a subject (e.g., a mammalian subject, e.g., a human subject).

In some embodiments, the biological sample is from a vertebrate animal (e.g., mammal, bird, fish, reptile, or amphibian). In some embodiments, the biological sample is from a human (e.g., the subject is a human). In other embodiments, the biological sample is from a non-human animal (e.g., the subject is a non-human mammal). In embodiments, the non-human mammal is a non-human primate (e.g., monkeys, apes), an ungulate (e.g., cattle, buffalo, sheep, goat, pig, camel, llama, alpaca, deer, horses, donkeys), a carnivore (e.g., dog, cat), a rodent (e.g., rat, mouse), or a lagomorph (e.g., rabbit). In some embodiments, the biological sample is from a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In some embodiments, the biological sample is from is an invertebrate such as an arthropod (e.g., insects, arachnids, crustaceans), a nematode, an annelid, a helminth, or a mollusk.

### I. Lipid Nanoparticles

The compositions (e.g., macromolecules, pairs of macromolecules, polypeptides, nucleic acids, and compositions comprising the same), methods, and delivery systems provided by the present disclosure may employ any suitable carrier or delivery modality described herein, including, in certain embodiments, lipid nanoparticles (LNPs). Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can include one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy) propyl-I-0-(w-methoxy (polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

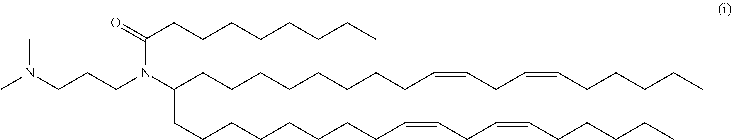 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver a polyribonucleotide (e.g., a circular polyribonucleotide, a linear polyribonucleotide) composition described herein to cells.

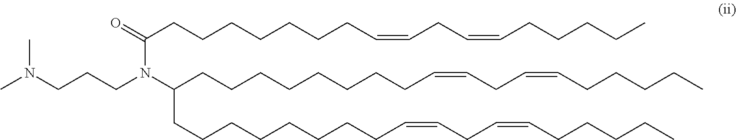 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP including Formula (ii) is used to deliver a polyribonucleotide (e.g., a circular polyribonucleotide, a linear polyribonucleotide) composition described herein to cells.

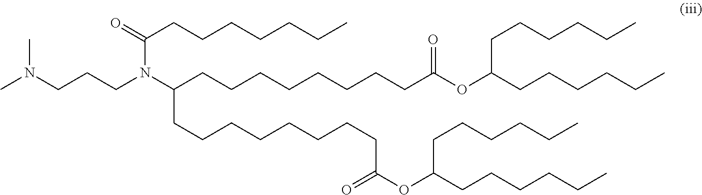 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP including Formula (iii) is used to deliver a polyribonucleotide (e.g., a circular polyribonucleotide, a linear polyribonucleotide) composition described herein to cells.

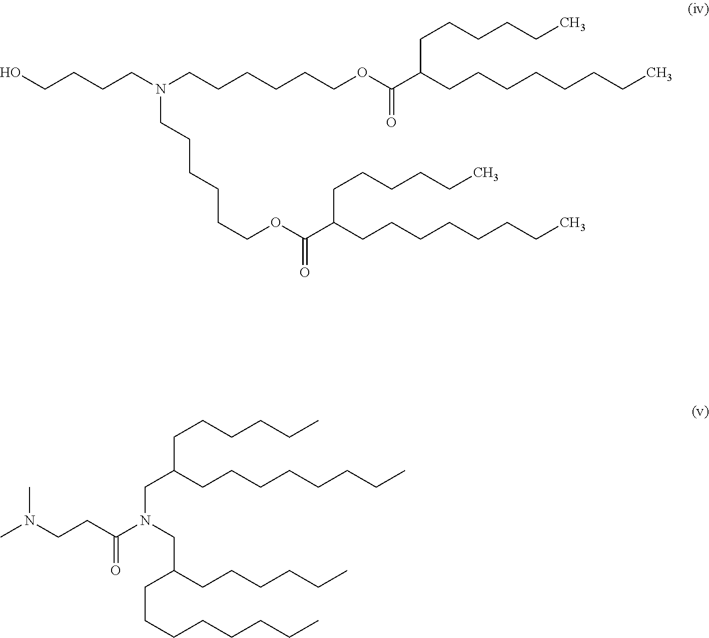

In some embodiments an LNP including Formula (v) is used to deliver a polyribonucleotide (e.g., a circular polyribonucleotide, a linear polyribonucleotide) composition described herein to cells.

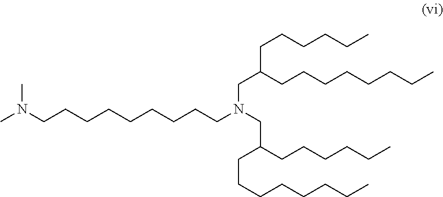 [SMILES: CCCCCCCCC(CCCCCC)CN(CCCCCCCCCN(C)C)CC(CCCCCC)CCCCCCCC]

In some embodiments an LNP including Formula (vi) is used to deliver a polyribonucleotide (e.g., a circular polyribonucleotide, a linear polyribonucleotide) composition described herein to cells.

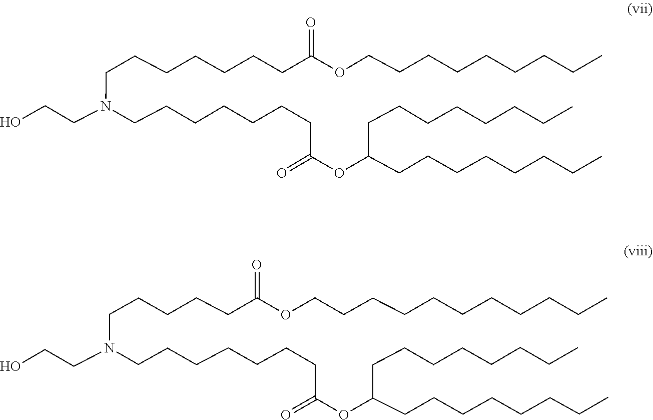 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP including Formula (viii) is used to deliver a polyribonucleotide (e.g., a circular polyribonucleotide, a linear polyribonucleotide) composition described herein to cells.

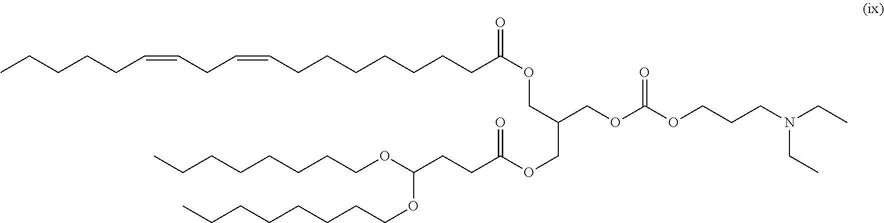 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP including Formula (ix) is used to deliver a polyribonucleotide (e.g., a circular polyribonucleotide, a linear polyribonucleotide) composition described herein to cells.

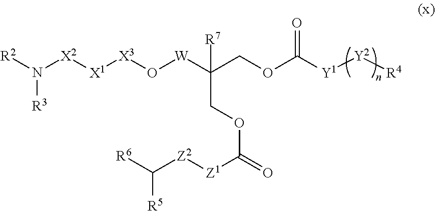

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

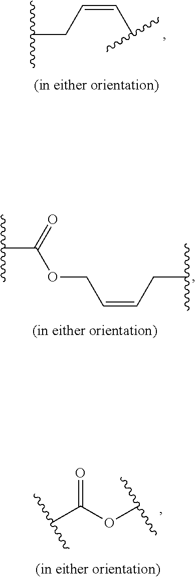 [SMILES: */C=C\C* ; *C/C=C\COC(*)=O ; *OC(*)=O]

n is 0 to 3, R⁴ is C₁₋₁₅ alkyl, Z¹ is C₁₋₆ alkylene or a direct bond,
Z² is

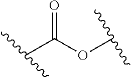 [SMILES: *OC(*)=O]

(in either orientation) or absent, provided that if Z¹ is a direct bond, Z² is absent;
R⁵ is C₅₋₉ alkyl or C₆₋₁₀ alkoxy, R⁶ is C₅₋₉ alkyl or C₆₋₁₀ alkoxy, W is methylene or a direct bond, and
R⁷ is H or Me, or a salt thereof, provided that if R³ and R² are C2 alkyls, X¹ is O, X² is linear C3 alkylene, X³ is C(=0), Y¹ is linear Ce alkylene, (Y²) n-R⁴ is

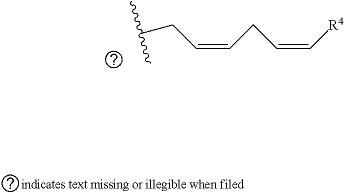

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (xii) is used to deliver a polyribonucleotide (e.g., a circular polyribonucleotide, a linear polyribonucleotide) composition described herein to cells.

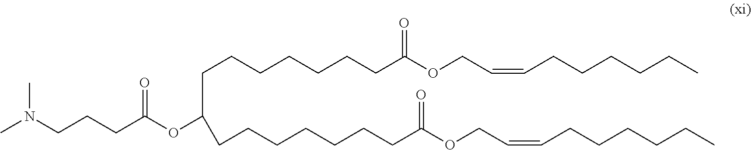 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver a polyribonucleotide (e.g., a circular polyribonucleotide, a linear polyribonucleotide) composition described herein to cells.

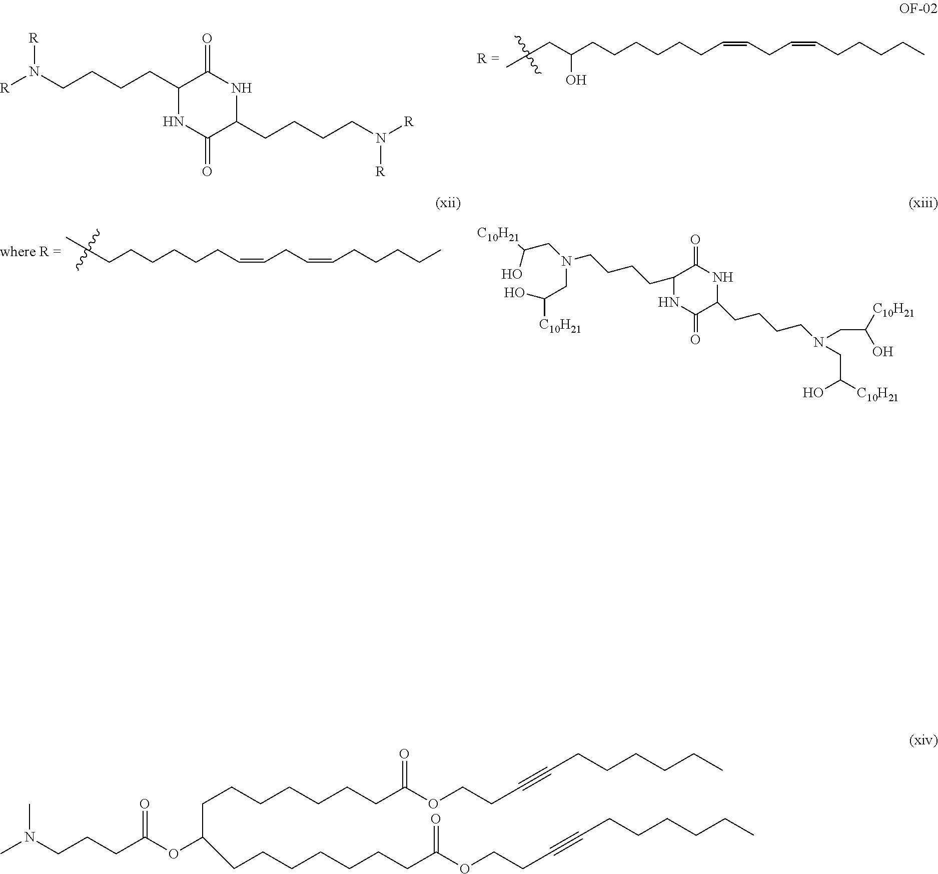

In some embodiments an LNP includes a compound of Formula (xiii) and a compound of Formula (xiv).

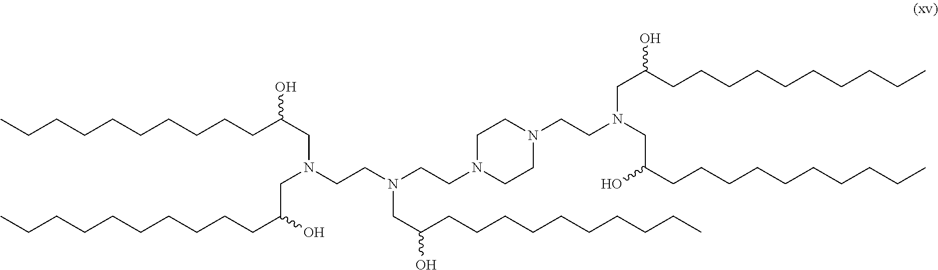 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP including Formula (xv) is used to deliver a polyribonucleotide (e.g., a circular polyribonucleotide, a linear polyribonucleotide) composition described herein to cells.

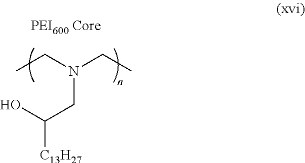

In some embodiments an LNP including a formulation of Formula (xvi) is used to deliver a polyribonucleotide (e.g., a circular polyribonucleotide, a linear polyribonucleotide) composition described herein to cells.

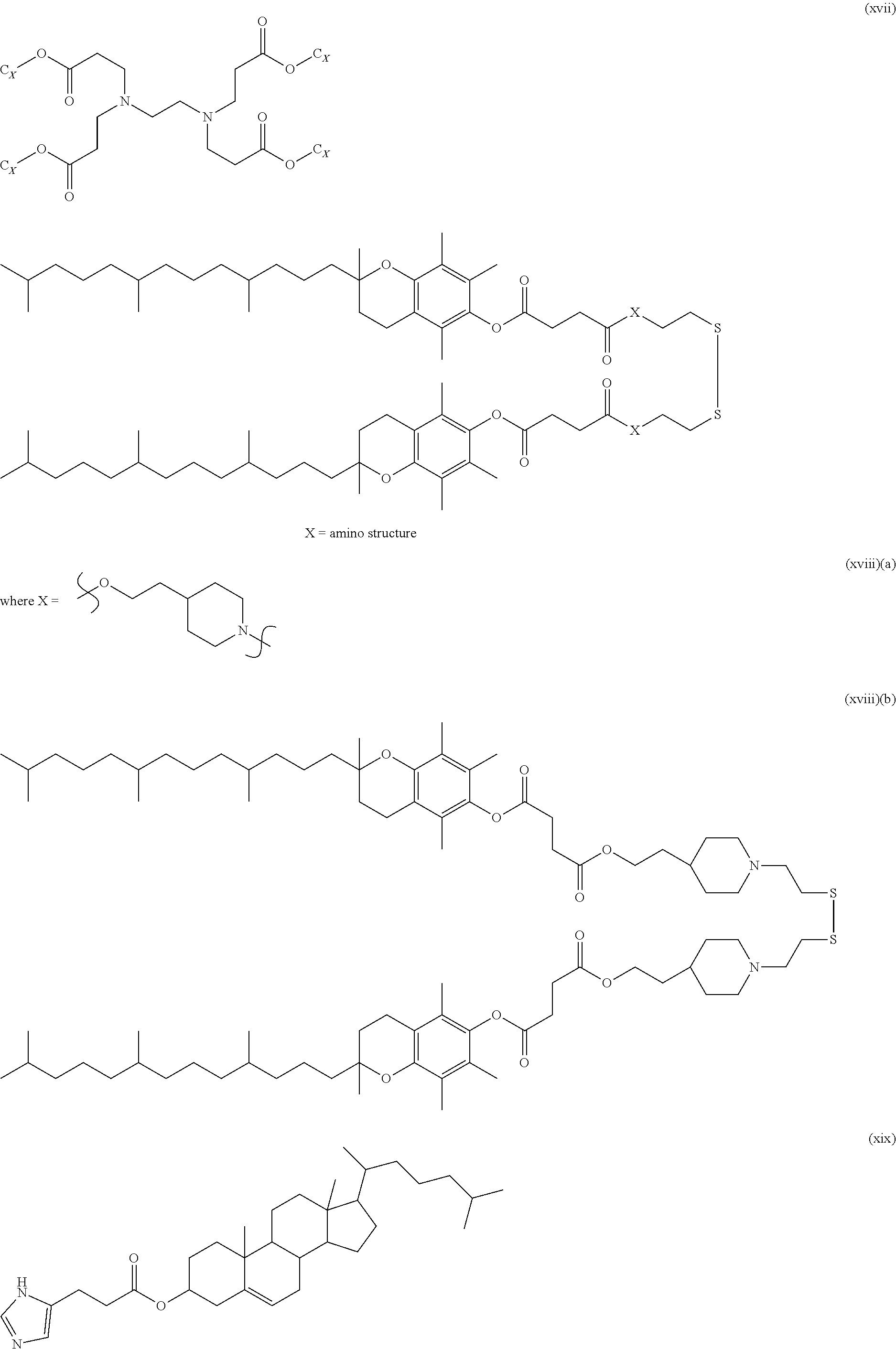

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

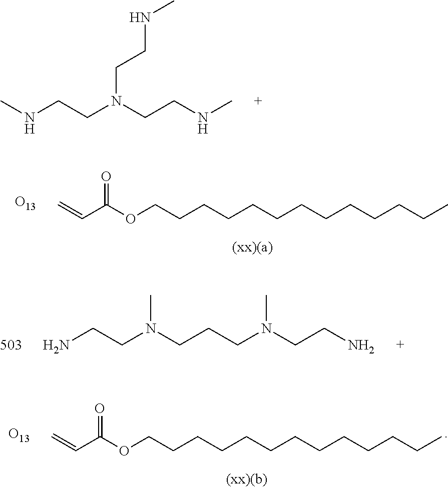

In some embodiments an LNP including Formula (xxi) is used to deliver a polyribonucleotide (e.g., a circular polyribonucleotide, a linear polyribonucleotide) composition described herein to cells. In some embodiments the LNP of Formula (xxi) is an LNP described by WO2021113777 (e.g., a lipid of Formula (1) such as a lipid of Table 1 of WO2021113777).

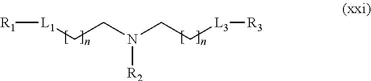

wherein
each n is independently an integer from 2-15; L₁ and L₃ are each independently —OC(O)—* or —C(O)O—*, wherein “*” indicates the attachment point to R₁ or R₃;
R₁ and R₅ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl) (alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl) (alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

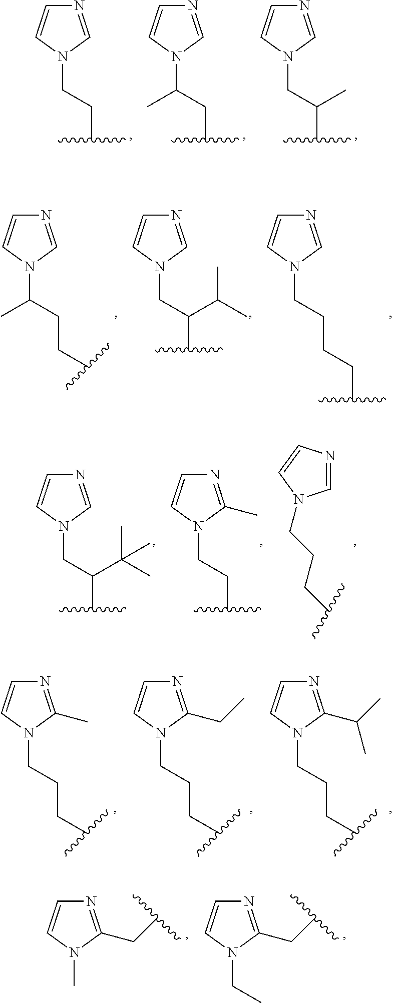

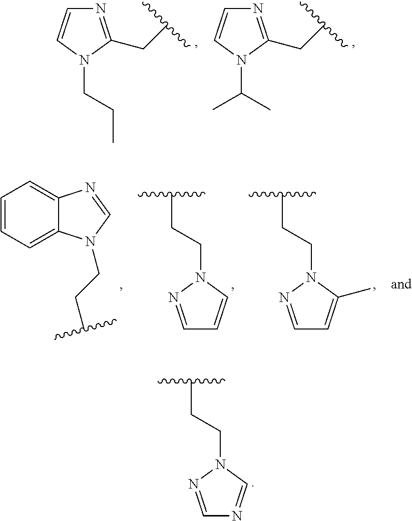

In some embodiments an LNP including Formula (xxii) is used to deliver a polyribonucleotide (e.g., a circular polyribonucleotide, a linear polyribonucleotide) composition described herein to cells. In some embodiments the LNP of Formula (xxii) is an LNP described by WO2021113777 (e.g., a lipid of Formula (2) such as a lipid of Table 2 of WO2021113777).

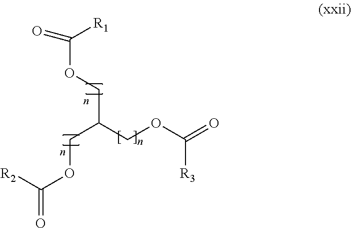

wherein
each n is independently an integer from 1-15;
R₁ and R₂ are each independently selected from a group consisting of:

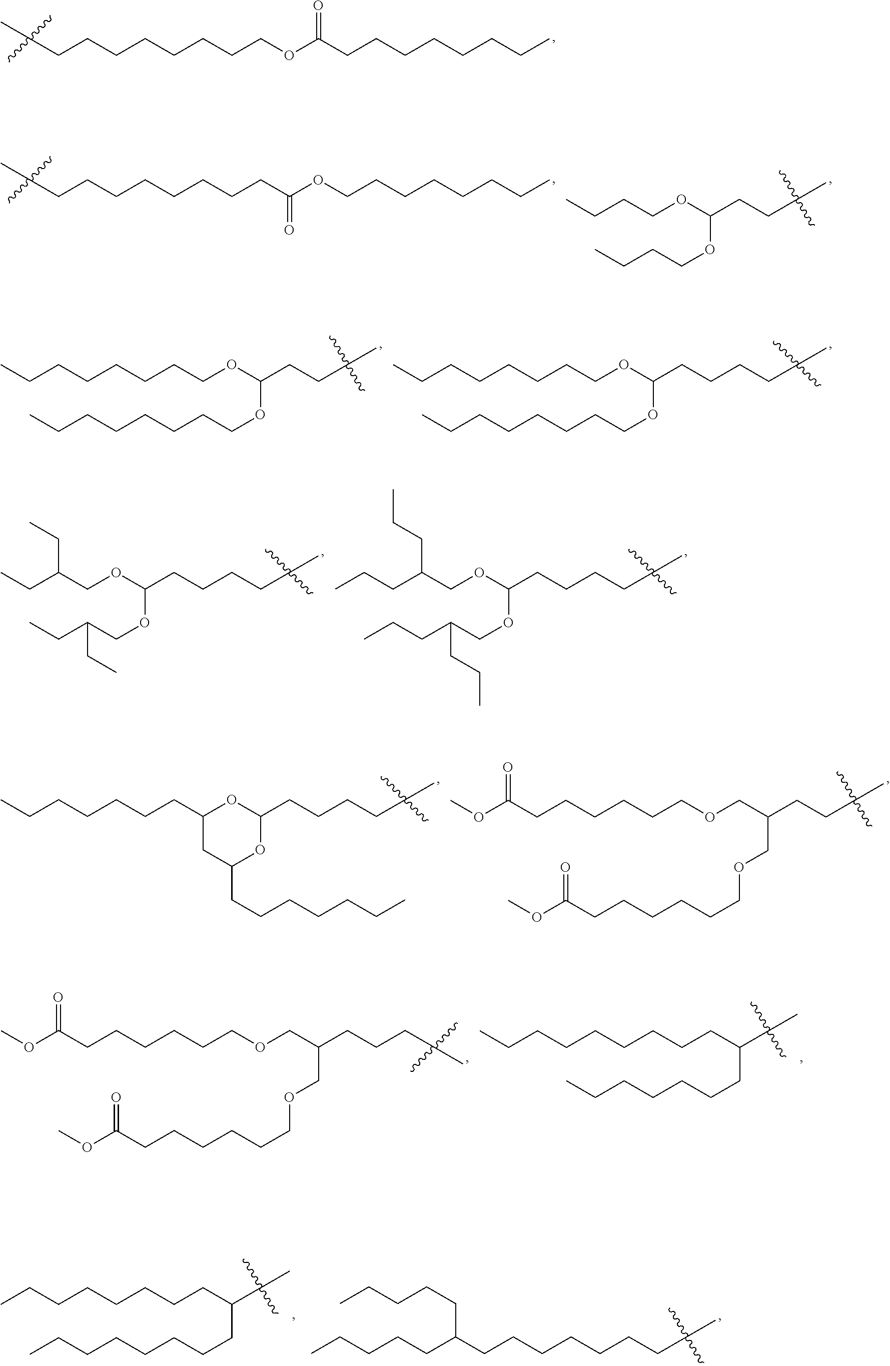

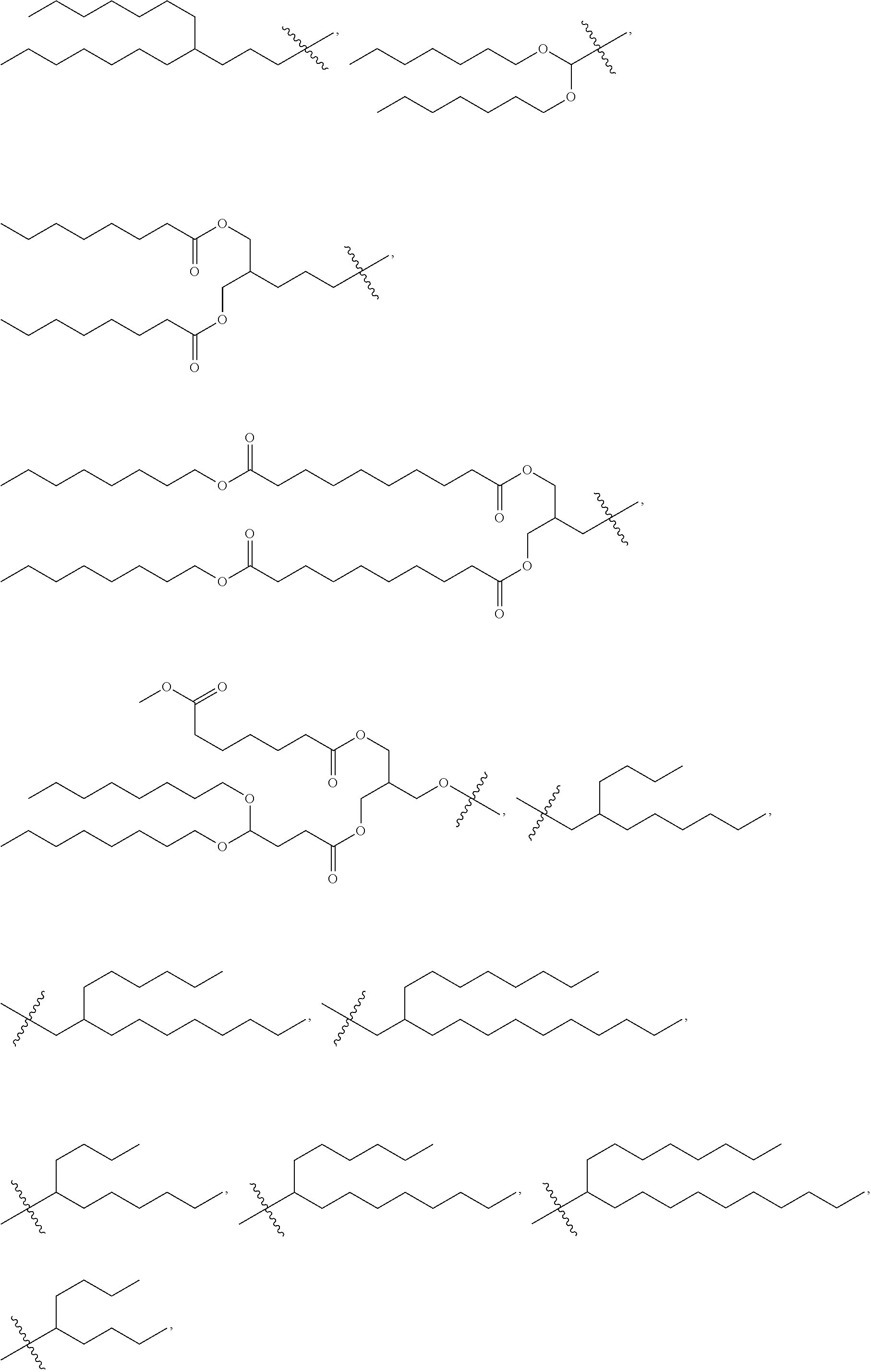

R₃ is selected from a group consisting of:

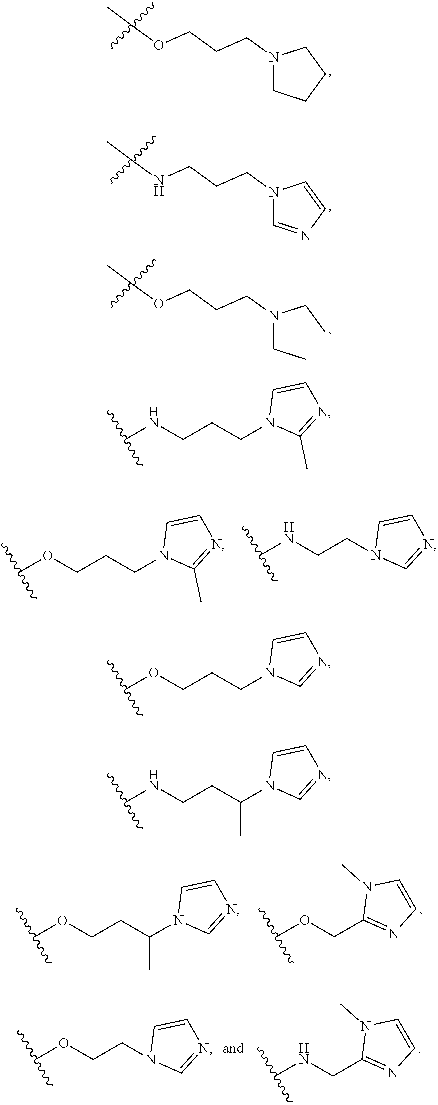

In some embodiments an LNP including Formula (xxiii) is used to deliver a polyribonucleotide (e.g., a circular polyribonucleotide, a linear polyribonucleotide) composition described herein to cells. In some embodiments the LNP of Formula (xxiii) is an LNP described by WO2021113777 (e.g., a lipid of Formula (3) such as a lipid of Table 3 of WO2021113777).

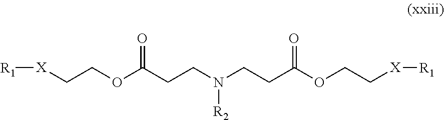

wherein
X is selected from —O—, —S—, or —OC(O)—*, wherein * indicates the attachment point to R₁;
R₁ is selected from a group consisting of:

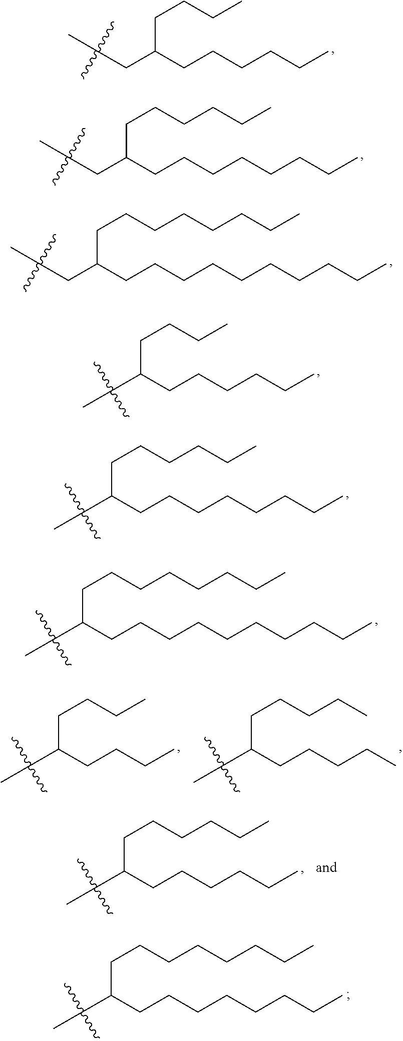

and R₂ is selected from a group consisting of:

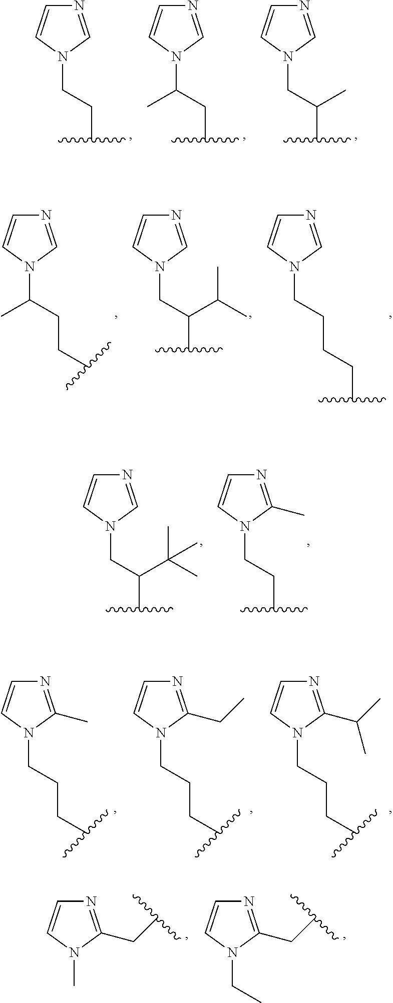

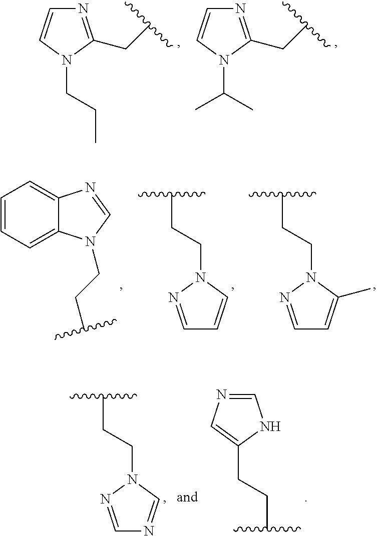

In some embodiments, a composition described herein (e.g., a nucleic acid (e.g., a circular polyribonucleotide, a linear polyribonucleotide) or a protein) is provided in an LNP that includes an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S,10R,13R,17R)-10,13-dimethyl-17-((R)-6-methylheptan-2-yl)-2, 3, 4, 7, 8, 9, 10, 11, 12, 13, 14, 15, 16, 17-tetradecahydro-IH-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; 1-5 or I-8 of WO2020/081938; 18 or 25 of U.S. Pat. No. 9,867,888; A of US2019/0136231; II of WO2020/219876; I of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3IZ)-heptatriaconta-6,9,28,3 I-tetraen-I9-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (I3Z,I6Z)-A,A-dimethyl-3-nonyldocosa-I3, I6-dien-I-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), monomethyl-phosphatidylethanolamine (such as 16-O-monomethyl PE), dimethyl-phosphatidylethanolamine (such as 16-O-dimethyl PE), I8-I-trans PE, I-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodecylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some aspects, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy) propyl-I-0-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (I-[8′-(Cholest-5-en-3 [beta]-oxy) carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy (polyethylene glycol)-2000]. In some embodiments, the PEG-lipid includes PEG-DMG, 1,2-dimyristoyl-sn-glycero-3-phosphoethanolamine-N-[methoxy (polyethylene glycol)-2000]. In some embodiments, the PEG-lipid includes a structure selected from:

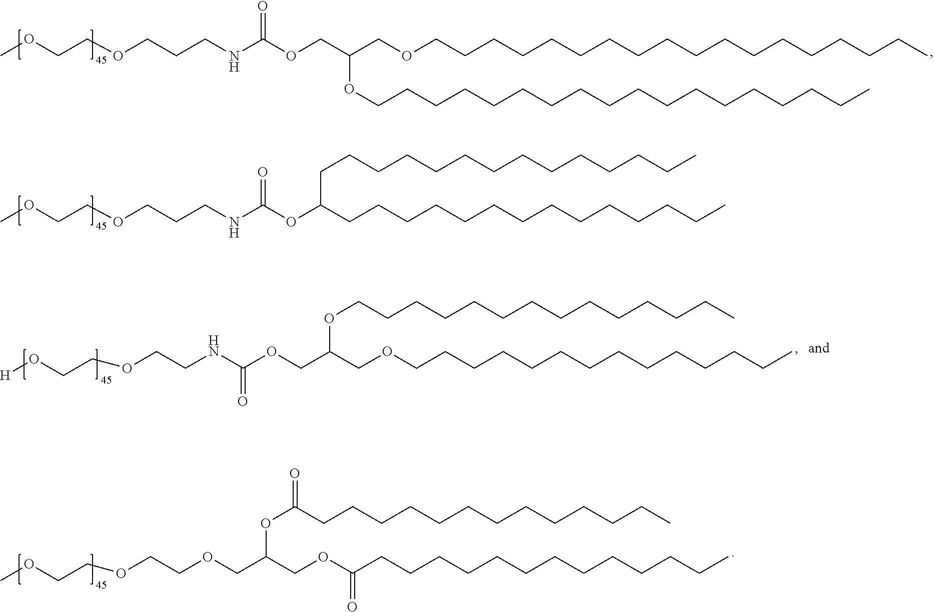

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,I2Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,I2Z)-octadeca-9,I2-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about I mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about-10 mV to about +20 mV, from about-10 mV to about +15 mV, from about-10 mV to about +10 mV, from about-10 mV to about +5 mV, from about-10 mV to about 0 mV, from about-10 mV to about-5 mV, from about-5 mV to about +20 mV, from about-5 mV to about +15 mV, from about-5 mV to about +10 mV, from about-5 mV to about +5 mV, from about-5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally include one or more coatings. In some embodiments, a LNP may be formulated in a capsule, film, or table having a coating. A capsule, film, or tablet including a composition described herein may have any useful size, tensile strength, hardness, or density.

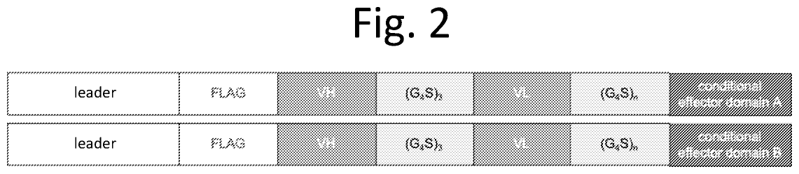

Additional exemplary lipids, formulations, methods, and characterization of LNPs are taught by WO2020/061457 and WO2021/113777, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51 (34): 8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA). Exemplary dosing of AAV including a polyribonucleotide (e.g., a circular polyribonucleotide, a linear polyribonucleotide) may include an MOI of about 10¹¹, 10¹², 10¹³, and 10¹⁴ vg/kg.

### II. Methods of Use

In some aspects, provided herein are methods (e.g., methods of inducing cellular effector functions, modulating the state of a cell, and/or treating disease) comprising providing any of the macromolecules or pair of macromolecules; nucleic acids or pairs of nucleic acids; or multimers described herein access to a cell.

### A. Methods of Inducing Cellular Effector Functions

In one aspect, provided herein is a method of inducing a cellular effector function in a cell, the method comprising contacting the cell with a macromolecule comprising a first binding domain linked to a second binding domain (e.g., a macromolecule as described in Section IA(i)), wherein (a) the first binding domain specifically binds a disease signature ligand in a biological sample comprising the cell (e.g., a disease signature ligand located on or in proximity to the cell); and (b) the second binding domain specifically binds an effector ligand in the biological sample (e.g., an effector ligand located on or in proximity to the cell) and induces the cellular effector function upon binding to the effector ligand; wherein the macromolecule is capable of forming a multimer in the presence of the disease signature ligand, and wherein induction of the effector function by the macromolecule is conditional upon each member of the multimer binding the disease signature ligand.

In another aspect, provided herein is a method of inducing a cellular effector function in a cell, the method comprising contacting the cell with a pair of macromolecules, each independently comprising a first binding domain linked to a second binding domain (e.g., a pair of macromolecules as described in Section IA(i)), wherein (a) the first binding domain of each macromolecule specifically binds a disease signature ligand in a biological sample comprising the cell (e.g., a disease signature ligand located on or in proximity to the cell); (b) the second binding domain of a first member of the pair of macromolecules specifically binds a first effector ligand in the biological sample (e.g., a first effector ligand located on or in proximity to the cell); and (c) the second binding domain of a second member of the pair of macromolecules specifically binds a second effector ligand in the biological sample (e.g., a second effector ligand located on or in proximity to the cell); wherein the second binding domain of the first member of the pair of macromolecules and the second binding domain of the second member of the pair of macromolecules induce the cellular effector function upon binding to the first effector ligand and the second effector ligand; wherein the pair of macromolecules is capable of forming a heteromultimer in the presence of the disease signature ligand, and wherein induction of the effector function by the macromolecule is conditional upon each member of the heteromultimer binding the disease signature ligand.

In another aspect, provided herein is a method of inducing a cellular effector function in a cell, comprising providing any of the macromolecules or pairs of macromolecules; nucleic acids or pairs of nucleic acids; or multimers (e.g., homomultimers or heteromultimers) described herein access to the cell (e.g., wherein the disease signature ligand(s) and effector ligand(s) bound by the macromolecule or pair of macromolecules are present on or in proximity to the cell), thereby inducing the cellular effector function in the cell.

In some embodiments of any of the above aspects, the cell is in a subject and the macromolecule, nucleic acid, or multimer is administered in a therapeutically effective amount.

In some embodiments, the subject has, or is suspected of having, a disease or disorder characterized by abnormal levels of the disease signature target, optionally wherein the subject was previously determined to have abnormal levels of the disease signature target.

### B. Methods of Modulating the State of a Cell

In another aspect, provided herein is a method of modulating the state of a cell, comprising providing any of the macromolecules or pairs of macromolecules; nucleic acids or pairs of nucleic acids; or multimers (e.g., homomultimers or heteromultimers) described herein access to the cell (e.g., wherein the disease signature ligand(s) and effector ligand(s) bound by the macromolecule or pair of macromolecules are present on or in proximity to the cell), thereby modulating the state of the cell. In some embodiments of any of the above aspects, the cell is in a subject and the macromolecule, nucleic acid, or multimer is administered in a therapeutically effective amount.

In some embodiments, the subject has, or is suspected of having, a disease or disorder characterized by abnormal levels of the disease signature target, optionally wherein the subject was previously determined to have abnormal levels of the disease signature target.

### C. Methods of Determining the State of a Cell

In another aspect, provided herein is a method of determining the state of a cell, comprising providing any of the macromolecules or pairs of macromolecules comprising a reporter domain; nucleic acids or pairs of nucleic acids encoding the same; or multimers (e.g., homomultimers or heteromultimers) of macromolecules comprising a reporter domain described herein access to the cell (e.g., wherein the disease signature ligand(s) and effector ligand(s) bound by the macromolecule or pair of macromolecules are present on or in proximity to the cell), and detecting the presence of the reporter domain, thereby determining the state of the cell.

In some embodiments of any of the above aspects, the cell is in a subject and the macromolecule, nucleic acid, or multimer is administered in a therapeutically effective amount.

In some embodiments, the subject has a disease or disorder characterized by abnormal levels of the disease signature target.

### EXAMPLES

The following are examples of the methods of the invention. It is understood that various other embodiments may be practiced, given the general description provided above.

Table of Contents (Examples):

| | |
|---|---|
| Example 1. | Protein complex modulating TPO receptor activity |
| | conditional on TGF-beta1 |
| Example 2. | Protein complex agonizing IL-2R activity |
| | conditional on TGF-beta1 or IL-8 |
| Example 3. | Protein complex agonizing IL-10R activity |
| | conditional on IFN-gamma or IL-6 |
| Example 4. | Fusion protein activating T lymphocyte cells |
| | conditional on TGF-beta1 or IL-8 |

### Example 1. Protein Complex Modulating TPO Receptor Activity Conditional on TGF-Beta1 or IL-8

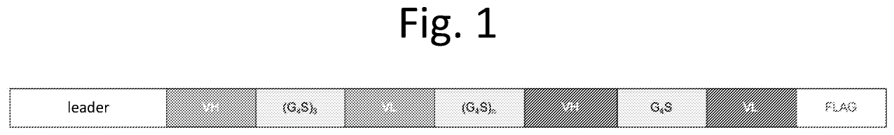

This Example describes the expression, purification, and characterization of (i) a protein complex that modulates the thrombopoietin (TPO) receptor conditional on the presence of transforming growth factor beta 1 (TGF-beta1) and (ii) a protein complex that modulates the TPO receptor conditional on the presence of interleukin-8 (IL-8). A genericized version of the fusion protein is shown in FIG. 1.

### A. Fusion Proteins

### TGF-Beta1-TPO Fusion Protein

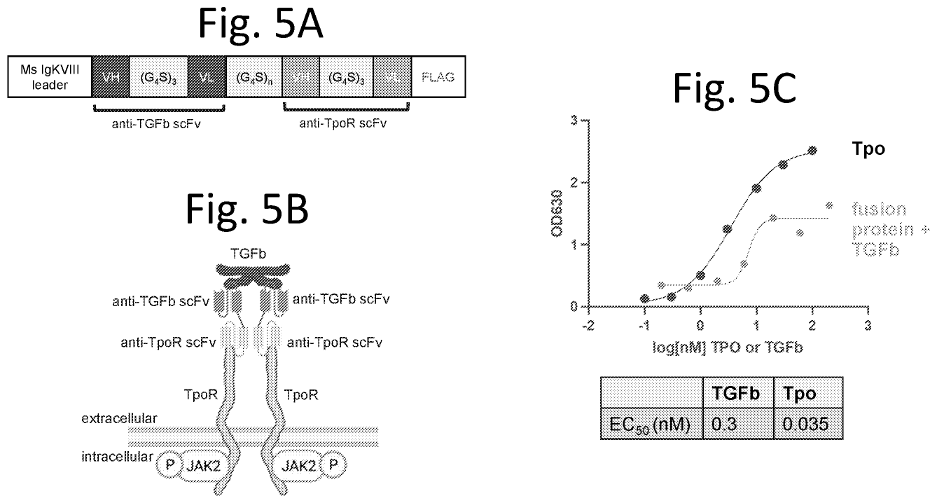

This Example provides a protein complex composed of two identical fusion proteins that modulate the TPO receptor conditional on the presence of TGF-beta1 (TGFb) (referred to herein as the “TGFb-TPO fusion protein”). Each fusion protein includes (from N- to C-terminus): a mouse immunoglobulin kappa variable 3 (IgKVIII) leader peptide (UniProt ID A0A140T8P0 positions M1 to G20); an anti-TGF-beta1 single-chain variable fragment (scFv) (PDB 4KV5; SEQ ID NOs: 13 and 14; Table 2); a (G₄S)ₙ linker, where n=2, 3, or 5; and an anti-TPO receptor heavy chain variable domain (VH) and light chain variable domain (VL) (SEQ ID NO: 1 and SEQ ID NO: 2 or SEQ ID NO: 10 and SEQ ID NO: 11, respectively; Table 2) joined by a G₄S linker (FIG. 5A). The proposed mechanism of action of the construct is shown in FIG. 5B: TGFb recruits two copies of the fusion protein via binding their TGFb-targeting domains, thus positioning the TpoR effector domains in close proximity to bind to and induce TpoR activity.

Optionally, each fusion protein further includes a FLAG affinity tag following the anti-TPO receptor VH and VL.

### IL-8-TPO Fusion Protein

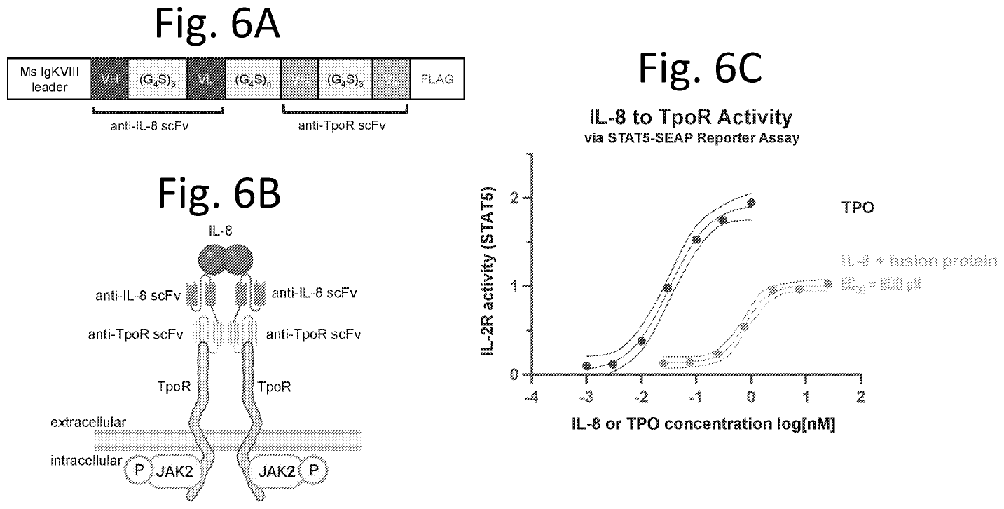

This Example also provides a protein complex composed of two identical fusion proteins that modulate the TPO receptor conditional on the presence of IL-8 (referred to herein as the “IL-8-TPO fusion protein”). Each fusion protein includes (from N- to C-terminus): a mouse immunoglobulin kappa variable 3 (IgKVIII) leader peptide (UniProt ID A0A140T8P0 positions M1 to G20); an anti-IL-8 scFv (PDB 6WZM, SEQ ID NO: 12; Table 2); a (G₄S)ₙ linker, where n=2, 3, or 5; and an anti-TPO receptor heavy chain variable domain (VH) and light chain variable domain (VL) (SEQ ID NO: 1 and SEQ ID NO: 2 or SEQ ID NO: 10 and SEQ ID NO: 11, respectively; Table 2) joined by a G₄S linker (FIG. 6A). The proposed mechanism of action of the construct is shown in FIG. 6B: IL-8 recruits two copies of the fusion protein via binding their IL-8-targeting domains, thus positioning the TpoR effector domains in close proximity to dimerize and induce TpoR activity.

Optionally, each fusion protein further includes a FLAG affinity tag following the anti-TPO receptor VH and VL.

### Expression and Purification

The fusion proteins are expressed via transient transfection of human embryonic kidney 293 (HEK293) cells under control of the cytomegalovirus (CMV) promoter. Secreted protein is analyzed by Western blot or ELISA using an anti-FLAG capture antibody to assess expression and quality. Fusion proteins are purified if necessary using a FLAG capture step, followed by a size exclusion chromatography (SEC) polishing step. Expression is expected to yield a strong band at the expected molecular weight by western blot, with minimal product-related variants.

### B. In Vitro Assays to Assess Effect of Fusion Protein on TPO Signaling Pathway in Presence or Absence of Soluble TPO or TGF-Beta1

HEK-BLUE™ TPO cells (InvivoGen catalog code: hkb-tpo) are used to evaluate activity of the fusion protein, using the manufacturer's prescribed protocol unless otherwise indicated. Briefly, HEK-BLUE™ TPO cells are transiently transfected with the fusion protein expression construct (as described in Example 1A) to secrete the fusion protein as autocrine in the assay. Alternatively, supernatant from HEK293 cells transiently expressing the fusion proteins is applied to the HEK-BLUE™ TPO cells as the test sample. Confirmatory assays are performed using FLAG-purified fusion protein material. JAK2/STAT5 pathway activation via secreted alkaline phosphatase (SEAP) production is compared among positive controls (administration of purified recombinant human TPO; transient transfection to express and secrete human TPO as an autocrine), negative controls (administration of recombinant human interleukin-2 (IL-2); mock transfection plasmid), and test samples (fusion protein variants). HEK-BLUE™ TPO cells expressing the fusion proteins are incubated overnight in the presence or absence of TPO, as well as in the presence or absence of TGF-beta1 (for the TGFb-TPO fusion protein) or IL-8 (for the IL-8-TPO fusion protein). Functional fusion protein samples are further evaluated by performing dose titrations of the fusion protein samples in the presence of TGF-beta1 or IL-8 to assess EC50, or in the presence of TGF-beta1 and TPO or IL-8 to assess IC50. The fusion proteins are expected to activate TPO receptor signaling conditional on the presence of TGF-beta1 (TGFb-TPO fusion protein) or IL-8 (IL-8-TPO fusion protein).

FIG. 5C shows a dose-response relationship between TGFb concentration and activity of the TGFb-TPO fusion protein (comprising the anti-TPO receptor VH and VL sequences of SEQ ID NO: 10 and SEQ ID NO: 11, respectively). TpoR SEAP reporter cells were stimulated overnight with a constant amount of the TGFb-TPO fusion protein and varying concentrations of TGFb, followed by measurement of SEAP activity in the culture supernatant.

FIG. 6C shows a dose-response relationship between IL-8 concentration and activity of the IL-8-TPO fusion protein (comprising the anti-TPO receptor VH and VL sequences of SEQ ID NO: 10 and SEQ ID NO: 11, respectively). TpoR SEAP reporter cells were stimulated overnight with a constant amount of the TGFb-TPO fusion protein and varying concentrations of IL-8, followed by measurement of SEAP activity in the culture supernatant.

### C. Characterization of TPO Receptor Agonism by Fusion Protein Versus Native Agonist

Cell samples for profiling and comparing gene expression between cells treated with the fusion proteins or soluble ligands are collected following the overnight incubation step of the assay described in Example 1B. RNA is extracted and purified according to the Qiagen RNeasy kit (Cat. No. 74104) with a threshold RNA Quality Number >7. RNA libraries for gene expression profiling are prepared using the Roche KAPA HyperPrep kit and sequenced on an Illumina NextSeq to generate 40-nucleotide paired-end reads. Sequenced reads are checked for quality, aligned, counted, and analyzed using standard procedures. See, for example, Kukurba and Montgomery, RNA sequencing and analysis. Cold Spring Harbor Protocols, 2015 (11), doi: 10.1101/pdb.top084970.

TABLE 2
Sequences

| SEQ ID | | |
|---|---|---|
| NO: | Name | Amino acid sequence |
| SEQ ID | anti-human TPO | DVQLVQSGAEVKKPGASVKVSCKASGFTFDNYAMHWVRQ |
| NO: 1 | receptor, VH domain | APGQGLEWIGYISWNSGDINYADSVKGRFTITTDKSTSTAYM |
| | | ELSSLRSEDTATYYCARDAGFGEFHYGLDVWGQGTTVTVS |
| | | S |
| SEQ ID | anti-human TPO | EVQLVESGGGLVQPGRSLRLSCATSGFTFDNYAMYWVRQA |
| NO: 10 | receptor, VH domain | PGKGLEWVSGISWNSGDIGYADSVKGRFTISRDNAKNSLYL |
| | | QMNSLRAEDTALYYCARDAGFGEFHYGLDVWGQGTTVTVS |
| | | S |
| SEQ ID | anti-human TPO | DIVLTQSPATLSLSPGERATLSCRASQGISSAMNWYQQKPG |
| NO: 2 | receptor, VL domain | KAPKRWIYDASKVASGVPARFSGSGSGTDYSLTINSLEAED |
| | | AATYYCQQFNSYPWTFGGGTKVEIK |
| SEQ ID | anti-human TPO | AIQLTQSPSSLSASVGDRVTITCRASQGISSALAWYQQKPGK |
| NO: 11 | receptor, VL domain | VPKLLIYDASSLESGVPSRFSGSGSGTDFTLTISSLQPEDFAT |
| | | YYCQQFNSYPWTFG QGTKVEIKR |
| SEQ ID | IL-2 conditional | PKKKIQLHAEHALYDALMILNIVKTNS |
| NO: 3 | effector domain A | |
| SEQ ID | IL-2 conditional | TNSPPAEEKLEDYAFNFELILEEIARLFESGDQKDEAEKAKR |
| NO: 4 | effector domain B | MKEWMKRIKTTASEDEQEEMANAIITILQSWIFS |
| SEQ ID | anti-IFN-gamma scFv | EVQLLESGGGLVQPGGSLRLSCAASGFTFSSYAMSWVRQA |
| NO: 5 | | PGKGLEWVSAISGSGGSTYYADSVKGRFTISRDNSKNTLYL |
| | | QMNSLRAEDTAVYYCAKDGSSGWYVPHWFDPWGQGTLVT |
| | | VSSGGGGSGGGGSGGGGSNFMLTQPHSVSESPGKTVTIS |
| | | CTRSSGSIASNYVQWYQQRPGSSPTTVIYEDNQRPSGVPD |
| | | RFSGSIDSSSNSASLTISGLKTEDEADYYCQSYDGSNRWMF |
| | | GGGTKLTVL |
| SEQ ID | anti-IL-10a, VHH | QVQLQESGGGSVQAGGSLRLSCAASGYSNCSYDMTWYRQ |
| NO: 6 | | APGKEREFVSAIHSDGSTRYADSVKGRFFISQDNAKNTVYL |
| | | QMNSLKPEDTAMYYCKTDPLHCRAHGGSWYSVRANYWGQ |
| | | GTQVTVSS |
| SEQ ID | anti-IL-10b, | QVQLQESGGGSVEAGGSLRLSCAASGYTHSSYCMGWFRQ |
| NO: 7 | VHH | APGKEREGVAAIDVDGSTTYADSVKGRFTISKDNAKNTLYLQ |
| | | MNSLKPEDTGMYYCAAEFADCSSNYFLPPGAVRYWGQGT |
| | | QVTVSS |
| SEQ ID | anti-CD3, | DVQLVQSGAEVKKPGASVKVSCKASGYTFTRYTMHWVRQA |
| NO: 8 | VH domain | PGQGLEWIGYINPSRGYTNYADSVKGRFTITTDKSTSTAYME |
| | | LSSLRSEDTATYYCARYYDDHYCLDYWGQGTTVTVSS |
| SEQ ID | anti-CD3, | DIVLTQSPATLSLSPGERATLSCRASQSVSYMNWYQQKPGK |
| NO: 9 | VL domain | APKRWIYDTSKVASGVPARFSGSGSGTDYSLTINSLEAEDA |
| | | ATYYCQQWSSNPLTFGGGTKVEIK |
| SEQ ID | Anti-IL8 scFv | QVQLVQSGAEVKKPGASVKVSCKASGYEFTSYWIHWVRQA |
| NO: 12 | | PGQGLEWMGNISPNSGSANYNEKFKSRVTMTRDTSTSTVY |
| | | MELSSLRSEDTAVYYCAREGPYSYYPSREYYGSDLWGQGT |
| | | LVTVSSGGGGSGGGGSGGGGSEIVLTQSPATLSLSPGERA |
| | | TLSCRASQSISNNLHWYQQKPGQAPRLLIYYTSRSVSGIPAR |
| | | FSGSGSGTDFTLTISSLEPEDFAVYYCGQNNEWPEVFGGGT |
| | | KVEIK |
| SEQ ID | anti-TGFb scFv, VH | QVQLVQSGAEVKKPGSSVKVSCKASGYTFSSNVISWVRQA |
| NO: 13 | domain | PGQGLEWMGGVIPIVDIANYAQRFKGRVTITADESTSTTYME |
| | | LSSLRSEDTAVYYCASTLGLVLDAMDYWGQGTLVTVSS |
| SEQ ID | anti-TGFb scFv, VL | ETVLTQSPGTLSLSPGERATLSCRASQSLGSSYLAWYQQKP |
| NO: 14 | domain | GQAPRLLIYGASSRAPGIPDRFSGSGSGTDFTLTISRLEPED |
| | | FAVYYCQQYADSPITFGQGTRLEIK |
| SEQ ID | anti-IL-6 VHH A | QVQLVESGGGLVQPGGSLRLSCAASGFSLDYYGVGWFRQ |
| NO: 32 | | APGKEREGVSCISSSDGDTYYADSVKGRFTISRDNAKNTVY |
| | | LQMNSLKPEDTAVYYCATDLSDYGVCSRWPSPYDYWGQG |
| | | TQVTVSS |
| SEQ ID | Anti-IL6 VHH B | AVQLVDSGGGLVQAGGSLRLSCAASGRFTSSSPMGWFRQ |
| NO: 33 | | APGKEREFVAAISGRSGNTYYADSVKGRFTISRDNAKNTVYL |
| | | QMNSLKPEDTAVYYCAAERVGLLLTWVAEGYDYWGQGTQV |
| | | TVSS |

### Example 2. Protein Complex Agonizing IL-2R Activity Conditional on TGF-Beta1 or IL-6

This Example describes the expression, purification, and characterization of (i) a protein complex that agonizes human interleukin-2 receptor (IL-2R) activity conditional on TGF-beta1 and (ii) a protein complex that agonizes human IL-2R activity conditional on interleukin-8 (IL-8; also known as CXCL8). A genericized version of the pair of fusion proteins is shown in FIG. 2.

### A. Fusion Proteins

### TGF-Beta1-IL-2R Fusion Protein Pair

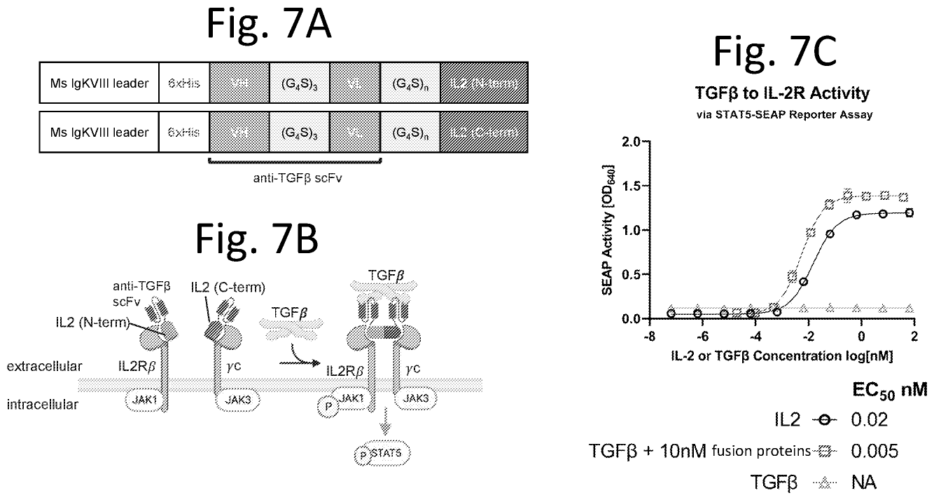

This Example provides a protein complex composed of two complementary fusion proteins (fusion proteins (a) and (b)) that modulates the IL-2 receptor (IL-2R) conditional on the presence of TGF-beta1. Each fusion protein includes (from N- to C-terminus): a mouse IgKVIII leader peptide (UniProt ID A0A140T8P0 positions M1 to G20); an anti-TGF-beta1 scFv (PDB 4KV5; SEQ ID NOs: 13 and 14; Table 2); a (G₄S)ₙ linker, where n=10, 15, or 25; and (a) conditional IL-2R effector domain A (SEQ ID NO: 3; Table 2) or (b) conditional IL-2R effector domain B (SEQ ID NO: 4; Table 2) (FIG. 7A). The proposed mechanism of action of the complementary fusion proteins is shown in FIG. 7B: TGFb recruits each of the fusion proteins via binding their TGFb-targeting domains, thus positioning the IL-2R effector domains in close proximity to dimerize and induce IL-2R activity.

Optionally, the fusion proteins each also include a FLAG affinity tag between the leader peptide and the anti-TGF-beta1 scFv.

### IL-8-IL-2R Fusion Protein Pair

This Example also provides a protein complex composed of two complementary fusion proteins that modulate the IL-2 receptor conditional on the presence of IL-8, wherein the two complementary fusion proteins each comprise an anti-IL-8 scFv (PDB 6WZM, SEQ ID NO: 12; Table 2).

### Expression and Purification

The fusion proteins are expressed and purified as described in Example 1A by 1) transfecting with equal quantities of each complementary construct (fusion proteins (a) and (b)) or 2) independently transfecting, purifying, and pre-mixing equal quantities of fusion proteins (a) and (b).

### B. In Vitro Assays for IL-2R Agonism on Cells in Presence or Absence of TGF-Beta1

HEK-BLUE™ CD122/CD132 cells (InvivoGen catalog code: hkb-il2bg) are used to evaluate fusion protein activity, using the manufacturer's prescribed protocol unless otherwise indicated and as described in Example 1B. IL-2 signaling pathway activation via SEAP production is compared among positive controls (administration of purified recombinant human IL-2; transient transfection to express and secrete human IL-2 as an autocrine), negative controls (administration of recombinant human TPO; mock transfection plasmid), and test samples (fusion proteins (a) and (b) and variants thereof). HEK-BLUE™ CD122/CD132 cells expressing fusion proteins are incubated overnight in the presence or absence of IL-2, as well as in the presence or absence of TGF-beta1. Functional fusion protein samples are further evaluated by performing dose titrations of the fusion protein samples in the presence of TGF-beta1 to assess EC50. Fusion proteins (a) and (b), administered in combination, are expected to activate IL-2 signaling conditional on the presence of TGF-beta1.

FIG. 7C shows a dose-response relationship between fusion proteins (a) and (b) and TGFb concentration. IL-2 reporter cells were stimulated overnight with a constant amount of fusion proteins (a) and (b) and varying concentrations of TGFb, followed by measurement of SEAP activity in the culture supernatant.

### C. Characterization of TGFR Agonism by Fusion Protein Versus Native Agonist

Transcriptomic comparison among fusion proteins and soluble ligands is performed as described in Example 1C.

### Example 3. Protein Complex Agonizing IL-10R Activity Conditional on IFN-Gamma or IL-6

This Example describes the expression, purification, and characterization of protein complexes that agonize the human interleukin 10 receptor (IL-10 receptor) conditional on the presence of interferon-gamma (IFN-gamma) or interleukin-6 (IL-6).

### A. Fusion Proteins

### IFN-Gamma-IL-10 Fusion Protein Pair

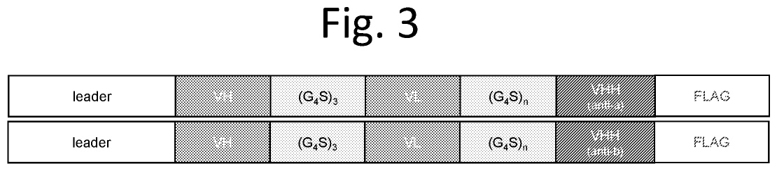

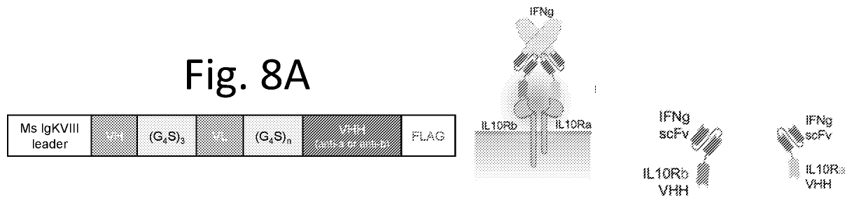

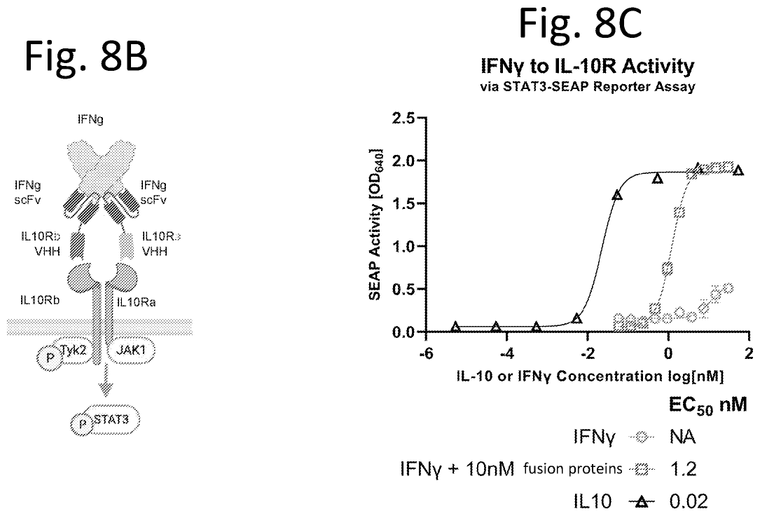

This Example provides a protein complex composed of two complementary fusion proteins (fusion proteins (c) and (d)) that modulates the IL-10 receptor conditional on the presence of IFN-gamma. Each fusion protein includes (from N- to C-terminus): a mouse IgKVIII leader peptide (UniProt ID A0A140T8P0 positions M1 to G20); an anti-IFN-gamma scFv (PDB 1T3F; SEQ ID NO: 5; Table 2); a (G₄S)ₙ linker, where n=1, 2, or 3; and an anti-IL10Ra VHH antibody (SEQ ID NO: 6; Table 2) (fusion protein (c)) or an anti-IL10Rb VHH antibody (SEQ ID NO: 7; Table 2) (fusion protein (d)) (FIG. 8A). A genericized version of the complementary fusion proteins is shown in FIG. 3. Optionally, each fusion protein further includes a FLAG affinity tag following the anti-IL10Ra VHH antibody (fusion protein (c)) or anti-IL10Rb VHH antibody (fusion protein (d)). The proposed mechanism of action of the construct is shown in FIG. 8B: IFN-gamma recruits each of the fusion proteins via binding their IFN-gamma-targeting domains, thus positioning the IL-10R-binding effector domains in close proximity to dimerize and induce IL-10R activity.

The fusion proteins are expressed and purified as described in Example 1A by 1) transfecting with equal quantities of each complementary construct (fusion proteins (c) and (d)) or 2) independently transfecting, purifying, and pre-mixing equal quantities of fusion proteins (c) and (d).

### IL-6-IL-10 Fusion Protein Pair

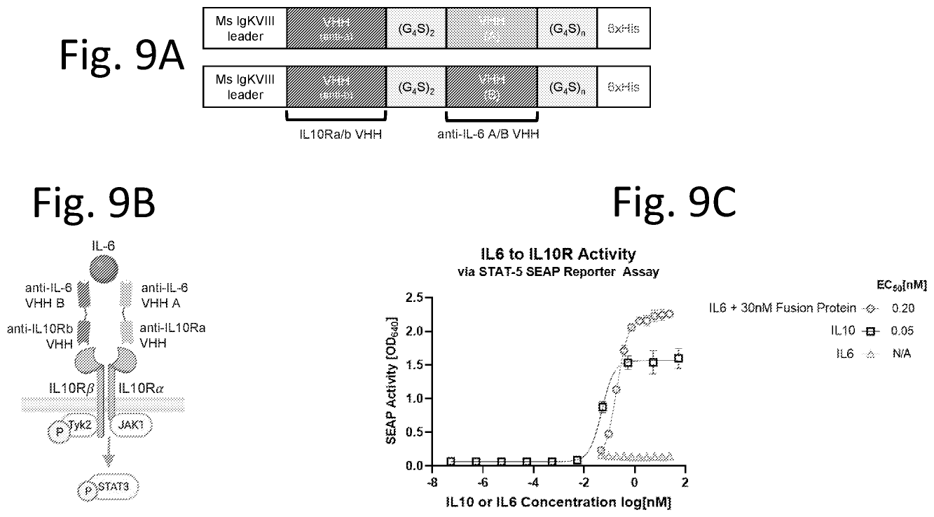

This Example also provides a protein complex composed of two complementary fusion proteins that modulate the IL-10 receptor conditional on the presence of IL-6. Each fusion protein includes (from N- to C-terminus): a mouse IgKVIII leader peptide (UniProt ID A0A140T8P0 positions M1 to G20); an anti-IL10Ra VHH antibody (first fusion protein) or an anti-IL10Rb VHH antibody (second fusion protein); a (G₄S)₂ linker; a first anti-IL-6 VHH antibody (SEQ ID NO: 32; first fusion protein) or a second anti-IL-6 VHH antibody (SEQ ID NO: 33; second fusion protein), wherein the first and second anti-IL-6 VHH antibodies bind distinct epitopes on IL-6; a (G₄S)ₙ linker; and a polyhistidine tag (FIG. 9A). Alternatively, the binding domains may be swapped such that (i) the first fusion protein comprises the first anti-IL-6 VHH antibody and the second anti-IL-6 VHH antibody (SEQ ID NO: 33) and (ii) the second fusion protein comprises the second anti-IL-6 VHH antibody and the first anti-IL-6 VHH antibody (SEQ ID NO: 32). The proposed mechanism of action of the construct is shown in FIG. 9B: IL-6 recruits each of the fusion proteins via binding their IL-6-targeting domains, thus positioning the IL-10R-binding effector domains in close proximity to dimerize and induce IL-10R activity.

### B. In Vitro Assays for IL-10 Agonism on Cells in Presence or Absence of IFN-Gamma or IL-6

HEK-BLUE™ IL-10 cells (InvivoGen catalog code: hkb-il10) are used to evaluate fusion protein activity, using the manufacturer's prescribed protocol unless otherwise indicated and as described in Example 1B. JAK1/STAT3 pathway activation via SEAP production is compared among positive controls (administration of purified recombinant human IL-10; transient transfection to express and secrete human IL-10 as an autocrine), negative controls (administration of recombinant human TPO; mock transfection plasmid), and test samples (fusion proteins or fusion protein pairs as described herein and variants thereof). HEK-BLUE™ IL-10 cells expressing fusion proteins are incubated overnight in the presence or absence of IL-10, or in the presence or absence of IFN-gamma (for the IFN-gamma-IL-10 fusion proteins) or IL-6 (for the IL-6-IL-10 fusion proteins). Functional fusion protein samples are further evaluated by performing dose titrations of the fusion protein samples in the presence of IFN-gamma or IL-6 to assess EC50, or in the presence of IFN-gamma or IL-6 and IL-10 to assess IC50. The fusion proteins or fusion protein pairs (administered in combination), are expected to activate IL-10 receptor signaling conditional on the presence of IFN-gamma (for the IFN-gamma-IL-10 fusion protein) or IL-6 (for the IL-6-IL-10 fusion proteins).

FIG. 8C shows a dose-response relationship between fusion proteins (c) and (d) and IFN-gamma concentration. IL-10 SEAP reporter cells were stimulated overnight with 10 nM of fusion proteins (c) and (d) and varying concentrations of IFNg, followed by measurement of SEAP activity in the culture supernatant.

FIG. 9C shows a dose-response relationship between IL-6 concentration and activity of the two complementary fusion proteins that modulate the IL-10 receptor conditional on the presence of IL-6. IL-10 SEAP reporter cells were stimulated overnight with a constant amount of the pair of fusion proteins and varying concentrations of IL-6, followed by measurement of SEAP activity in the culture supernatant.

### C. Characterization of IL-10 Agonism by Fusion Protein Versus Native Agonist

Transcriptomic comparison among fusion proteins and soluble ligands is performed as described in Example 1C.

### Example 4. Fusion Protein Activating T Lymphocyte Cells Conditional on TGF-Beta1 or IL-8

This Example describes the expression, purification, and characterization of protein complexes that activate T cells via cluster of differentiation 3 (CD3) conditional on the presence of TGF-beta1.

### A. Expression and Purification of Fusion Protein

### TGF-Beta1-CD3 Fusion Protein Pair

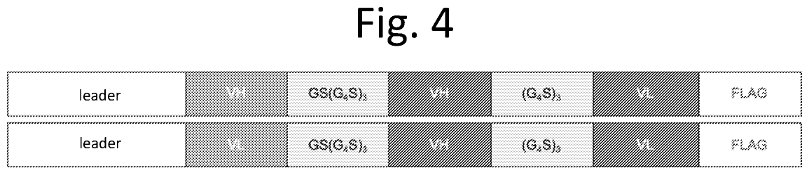

This Example provides a protein complex composed of two complementary fusion proteins (fusion proteins (e) and (f)) that activates T cells via CD3 conditional on the presence of TGF-beta1. Each fusion protein includes (from N- to C-terminus): a mouse IgKVIII leader peptide (UniProt ID A0A140T8P0 positions M1 to G20); an anti-CD3 VH domain of an scFv engineered to have low (KD>1 μM) affinity for its matching variable domain (SEQ ID NO. 8; Table 2) (fusion protein (e)) or an anti-CD3 VL domain of an scFv engineered to have low (KD>1 μM) affinity for its matching variable domain (SEQ ID NO. 9; Table 2); a GS(G₄S) linker; and an anti-TGF-beta1 scFv (PDB 4KV5; SEQ ID NOs: 13 and 14; Table 2). A generic version of the construct is shown in FIG. 4. Optionally, each fusion protein further includes a FLAG affinity tag following the anti-TGF-beta1 scFv.

The fusion proteins are expressed and purified as described in Example 1A by 1) transfecting with equal quantities of each complementary construct (fusion proteins (e) and (f)) or 2) independently transfecting, purifying, and pre-mixing equal quantities of fusion proteins (e) and (f).

### IL-8-CD3 Fusion Protein Pair

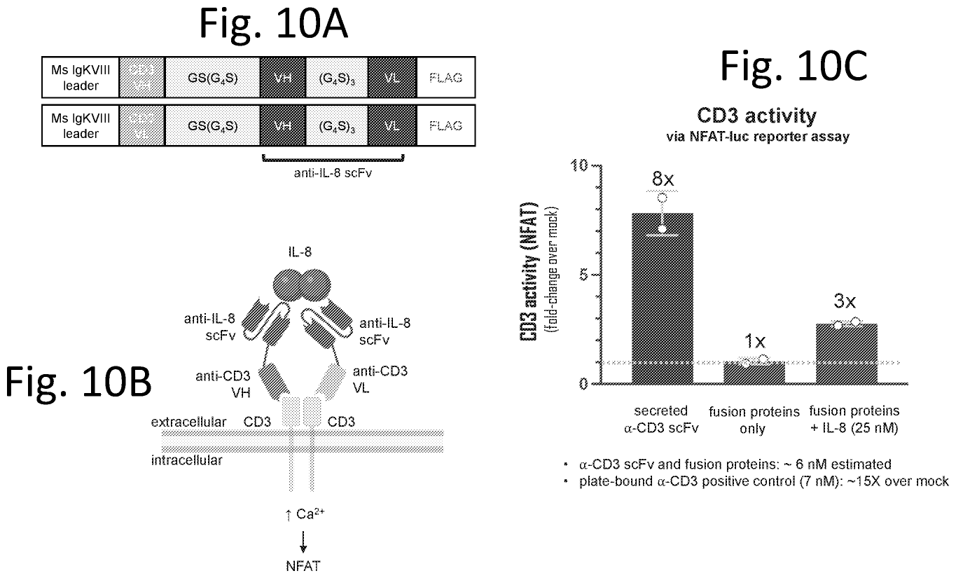

This Example provides a protein complex composed of two complementary fusion proteins that activates T cells via CD3 conditional on the presence of IL-8. Each fusion protein includes (from N- to C-terminus): a mouse IgKVIII leader peptide (UniProt ID A0A140T8P0 positions M1 to G20); an anti-CD3 VH domain of an scFv engineered to have low (KD>1 μM) affinity for its matching variable domain (SEQ ID NO. 8; Table 2) (first fusion protein) or an anti-CD3 VL domain of an scFv engineered to have low (KD>1 μM) affinity for its matching variable domain (SEQ ID NO. 9; Table 2) (second fusion protein); a GS(G₄S) linker; and an anti-IL-8 scFv (SEQ ID NO: 12; Table 2). A generic version of the construct is shown in FIG. 4; the construct of the present Example is shown in FIG. 10A. Optionally, each fusion protein further includes a FLAG affinity tag following the anti-TGF-beta1 scFv. The proposed mechanism of action of the construct is shown in FIG. 10B: IL-8 recruits each of the fusion proteins via binding their IL-8-targeting domains, thus positioning the CD3-binding effector domains in close proximity to dimerize and induce CD3 activity.

The fusion proteins are expressed and purified as described in Example 1A by 1) transfecting with equal quantities of each complementary construct (fusion proteins (e) and (f)) or 2) independently transfecting, purifying, and pre-mixing equal quantities of fusion proteins (e) and (f).

### B. In Vitro Assays for T-Cell Activation in Presence or Absence of TGF-Beta1 or IL-8

JURKAT-LUCIA™ nuclear factor of activated T-cells (NFAT) cells (InvivoGen catalog code: jktl-nfat) are used to evaluate fusion protein activity, using the manufacturer's prescribed protocol unless otherwise indicated and as described in Example 1B. NFAT pathway activation via luciferase production is compared among positive controls (administration of anti-CD3 and anti-CD28 scFvs), negative controls (administration of recombinant human TPO; mock transfection plasmid), and test samples (fusion proteins or fusion protein pairs as described herein and variants thereof). JURKAT-LUCIA™ NFAT cells expressing complementary pairs of fusion proteins are incubated overnight with an anti-CD28 antibody, as well as in the presence or absence of an anti-CD3 scFv or in the presence or absence of TGF-beta1 (for the TGF-beta1-CD3 fusion proteins) or IL-8 (for the IL-8-CD3 fusion proteins). Functional fusion protein pairs are further evaluated by performing dose titrations of the fusion protein pairs in the presence of anti-CD28 scFv+TGF-beta1 (or IL-8) to assess EC50, or anti-CD28 scFv+TGF-beta1 (or IL-8)+anti-CD3 scFv to assess IC50. The fusion proteins or fusion protein pairs (administered in combination), are expected to activate T cells conditional on the presence of TGF-beta1 (for the TGF-beta1-CD3 fusion proteins) or IL-8 (for the IL-8-CD3 fusion proteins).

FIG. 10C shows a dose-response relationship between fusion proteins (e) and (f) and IL-8 concentration. NFAT-luc Jurkat reporter cells were stimulated for 24 hours with a constant amount of the fusion proteins in the presence or absence of 25 nM IL-8, followed by measurement of luciferase activity in the culture supernatant.

### C. Characterization of T Cell Activation by Fusion Protein Versus Native Agonist

Transcriptomic comparison among fusion proteins and soluble ligands is performed as in Example 1C.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Other embodiments are within the claims.

What is claimed is:

1. A macromolecule comprising a first binding domain linked to a second binding domain, wherein:
(a) the first binding domain specifically binds a disease signature ligand in a biological sample; and
(b) the second binding domain specifically binds an effector ligand in the biological sample and induces a cellular effector function upon binding to the effector ligand;
wherein the macromolecule is capable of forming a multimer in the presence of the disease signature ligand, and wherein induction of the effector function by the macromolecule is conditional upon each member of the multimer binding the disease signature ligand.

2. The macromolecule of claim 1, wherein the multimer is a dimer, trimer, or tetramer.

3. The macromolecule of claim 2, wherein the multimer is a dimer.

4. The macromolecule of claim 3, wherein the dimer is a homodimer.

5. A pair of macromolecules, each independently comprising a first binding domain linked to a second binding domain, wherein:
(a) the first binding domain of each macromolecule specifically binds a disease signature ligand in a biological sample;
(b) the second binding domain of a first member of the pair of macromolecules specifically binds a first effector ligand in the biological sample; and
(c) the second binding domain of a second member of the pair of macromolecules specifically binds a second effector ligand in the biological sample;
wherein the second binding domain of the first member of the pair of macromolecules and the second binding domain of the second member of the pair of macromolecules induce a cellular effector function upon binding to the first effector ligand and the second effector ligand;
wherein the pair of macromolecules is capable of forming a heteromultimer in the presence of the disease signature ligand, and wherein induction of the effector function by the macromolecule is conditional upon each member of the heteromultimer binding the disease signature ligand.

6. A pair of macromolecules, each independently comprising a first binding domain linked to a second binding domain, wherein:
(a) the first member of the pair of macromolecules comprises a first binding domain 1 (FBD1) that specifically binds a first epitope of disease signature ligand in a biological sample;
(b) the second member of the pair of macromolecules comprises a first binding domain 2 (FBD2) that specifically binds a second epitope of disease signature ligand in a biological sample; and
(c) the second binding domain specifically binds an effector ligand in the biological sample and induces a cellular effector function upon binding to the effector ligand;
wherein the macromolecule is capable of forming a multimer in the presence of the disease signature ligand, and wherein induction of the effector function by the macromolecule is conditional upon each member of the multimer binding the disease signature ligand.

7. A pair of macromolecules, each independently comprising a first binding domain linked to a second binding domain, wherein:
(a) the first member of the pair of macromolecules comprises a first binding domain 1 (FBD1) that specifically binds a first epitope of disease signature ligand in a biological sample;
(b) the second member of the pair of macromolecules comprises a first binding domain 2 (FBD2) that specifically binds a second epitope of disease signature ligand in a biological sample;
(c) the second binding domain of the first member of the pair of macromolecules specifically binds a first effector ligand in the biological sample; and
(d) the second binding domain of the second member of the pair of macromolecules specifically binds a second effector ligand in the biological sample;
wherein the macromolecule is capable of forming a multimer in the presence of the disease signature ligand, and wherein induction of the effector function by the macromolecule is conditional upon each member of the multimer binding the disease signature ligand.

8. The macromolecule of claim 5 or 6, wherein the heteromultimer is a dimer.

9. The macromolecule or pair of macromolecules of any one of claims 1-8, wherein the disease signature ligand is a protein, a peptide, or a small molecule.

10. The macromolecule or pair of macromolecules of claim 9, wherein the protein is a soluble protein or an insoluble protein.

11. The macromolecule or pair of macromolecules of any one of claims 1-8, wherein the disease signature ligand is a cytokine.

12. The macromolecule or pair of macromolecules of claim 11, wherein the cytokine is an interleukin, an interferon, a growth factor, a chemokine, or a member of the TNF family.

13. The macromolecule or pair of macromolecules of claim 12, wherein the interleukin is IL-1, IL-2, IL-4, IL-6, IL-8, IL-10, IL-12, IL-15, IL-17, or IL-23; the interferon is IFN-gamma; the growth factor is transforming growth factor beta (TGF-beta), granulocyte colony stimulating factor (GCSF) granulocyte-macrophage colony-stimulating factor (GCSF), epidermal growth factor (EGF), or erythropoietin (EPO); the chemokine is monocyte chemoattractant protein-1 (MCP-1) or interferon gamma-induced protein 10 (IP-10); or the member of the TNF family is TNF-alpha.

14. The macromolecule or pair of macromolecules of any one of claims 1-8, wherein the disease signature ligand is a neurotransmitter.

15. The macromolecule or pair of macromolecules of any one of claims 1-8, wherein the disease signature ligand is a cell surface receptor, a surface antigen, a membrane-bound protein, an extracellular matrix component, or an integrin.

16. The macromolecule or pair of macromolecules of any one of claims 1-8, wherein the disease signature ligand is a self antigen of an organism from which the biological sample is derived.

17. The macromolecule or pair of macromolecules of claim 16, wherein the self antigen is an anti-drug antibody (ADA), an autoantibody, or a tumor marker.

18. The macromolecule or pair of macromolecules of any one of claims 1-8, wherein the disease signature ligand is a nucleic acid.

19. The macromolecule or pair of macromolecules of any one of claims 1-8, wherein the disease signature ligand is a carbohydrate, a lipid, a peptide, a nucleoside, or a combination of the foregoing.

20. The macromolecule or pair of macromolecules of any one of claims 1-8, wherein the disease signature ligand is a hormone, an amino acid derivative, a steroid, or an eicosanoid.

21. The macromolecule or pair of macromolecules of any one of claims 1-8, wherein the disease signature ligand is a non-self antigen.

22. The macromolecule or pair of macromolecules of claim 21, wherein the disease signature ligand is a virus, a bacterium, or a fragment or antigen thereof.

23. The macromolecule or pair of macromolecules of any one of claims 1-22, wherein the disease signature ligand is multimeric.

24. The macromolecule or pair of macromolecules of claim 23, wherein the disease signature ligand is dimeric, trimeric, or tetrameric.

25. The macromolecule or pair of macromolecules of any one of claims 1-24, wherein the first binding domain, FBD1, or FBD2 comprises a polypeptide that specifically binds the disease signature ligand.

26. The macromolecule or pair of macromolecules of claim 25, wherein the polypeptide is an antibody or a fragment thereof.

27. The macromolecule or pair of macromolecules of claim 26, wherein the antibody or fragment thereof is an scFv, a monospecific tandem scFv (taFv), a bispecific taFv, a VHH, a VNAR, a Fab, a monospecific single-chain diabody, a bispecific single-chain diabody, or a dual-affinity re-targeting antibody (DART).

28. The macromolecule or pair of macromolecules of claim 25, wherein the polypeptide is an antibody mimetic.

29. The macromolecule or pair of macromolecules of claim 28, wherein the antibody mimetic is an affibody, an affilin, an affimer, an affitin, an alphabody, an anticalin, a lipocalin, an avimer, a DARPin, a fynomer, a gastrobody, a knottin, a Kunitz domain peptide, a monobody, a fibronectin type III domain (FN3)-based binder, a nanoantibody, a nanoCLAMP, an optimer, a repebody, a pronectin, a centyrin, an obody, a peptide aptamer, a synthetic peptide, or a variable lymphocyte receptor (VLR).

30. The macromolecule or pair of macromolecules of claim 25, wherein the polypeptide is an endogenous binding domain.

31. The macromolecule or pair of macromolecules of claim 30, wherein the endogenous binding domain is a cell receptor domain, an enzyme domain, a variable lymphocyte receptor (VLR) domain, a receptor ectodomain, a nuclear hormone receptor ligand-binding domain, or a DNA-binding domain.

32. The macromolecule or pair of macromolecules of any one of claims 1-24, wherein the first binding domain, FBD1, or FBD2 comprises an oligonucleotide that specifically binds the disease signature ligand.

33. The macromolecule or pair of macromolecules of claim 32, wherein the oligonucleotide is a nucleic acid aptamer.

34. The macromolecule or pair of macromolecules of claim 33, wherein the nucleic acid aptamer is a DNA aptamer.

35. The macromolecule or pair of macromolecules of any one of claims 1-24, wherein the first binding domain, FBD1, or FBD2 comprises a chemical molecule that specifically binds the disease signature ligand.

36. The macromolecule or pair of macromolecules of any one of claims 1-35, wherein the first binding domain, FBD1, or FBD2 has affinity to two or more disease signature moieties.

37. The macromolecule or pair of macromolecules of any one of claims 1-36, wherein the effector ligand is a protein or a peptide.

38. The macromolecule or pair of macromolecules of any one of claims 1-37, wherein the effector ligand is a cell-surface receptor or an intracellular receptor.

39. The macromolecule or pair of macromolecules of claim 38, wherein the cell-surface receptor is a catalytic receptor or the intracellular receptor is a nuclear hormone receptor.

40. The macromolecule or pair of macromolecules of claim 39, wherein the catalytic receptor is a receptor tyrosine kinase (RTK), a receptor serine/threonine kinase (RSK), a type 1 cytokine receptor, a type 2 cytokine receptor, or a tumor necrosis factor (TNF) superfamily receptor.

41. The macromolecule or pair of macromolecules of claim 40, wherein the RTK is VEGFR, the RSK is TGFBR2, the type 1 cytokine receptor is IL-2R, the type 2 cytokine receptor is IL10R, or the TNF superfamily receptor is TNFR2 or 4-1BB.

42. The macromolecule of any one of claims 1-4, 6, and 8-41, wherein the second binding domain is an agonist of the effector ligand.

43. The macromolecule of claim 42, wherein the effector ligand must be homodimerized to exert a cellular effector function.

44. The macromolecule of claim 43, wherein the effector ligand is homodimerized in the presence of the multimer of the macromolecule.

45. The pair of macromolecules of any one of claims 5 and 7-39, wherein the first effector ligand and the second effector ligand must be associated to exert a cellular effector function.

46. The pair of macromolecules of claim 45, wherein the first effector ligand and the second effector ligand are associated in the presence of the multimer of the macromolecule.

47. The pair of macromolecules of claim 45 or 46, wherein the association is heterodimerization.

48. The macromolecule or pair of macromolecules of any one of claims 1-47, wherein the cellular effector function is a biological activity.

49. The macromolecule or pair of macromolecules of any one of claims 1-48, wherein the cellular effector function is a therapeutic activity.

50. The macromolecule or pair of macromolecules of any one of claims 1-48, wherein the cellular effector function is a disease activity.

51. The macromolecule or pair of macromolecules of any one of claims 1-50, wherein the second binding domain comprises a polypeptide that specifically binds the effector ligand.

52. The macromolecule or pair of macromolecules of claim 51, wherein the polypeptide is an antibody or a fragment thereof.

53. The macromolecule or pair of macromolecules of claim 52, wherein the antibody or fragment thereof is an scFv, a monospecific taFv, a bispecific taFv, a VHH, a VNAR, a Fab, a monospecific single-chain diabody, a bispecific single-chain diabody, or a DART.

54. The macromolecule or pair of macromolecules of claim 51, wherein the polypeptide is an antibody mimetic.

55. The macromolecule or pair of macromolecules of claim 54, wherein the antibody mimetic is an affibody, an affilin, an affimer, an affitin, an alphabody, an anticalin, a lipocalin, an avimer, a DARPin, a fynomer, a gastrobody, a knottin, a Kunitz domain peptide, a monobody, a FN3-based binder, a nanoantibody, a nanoCLAMP, an optimer, a repebody, a pronectin, a centyrin, an obody, a peptide aptamer, a synthetic peptide, or a VLR.

56. The macromolecule or pair of macromolecules of claim 51, wherein the polypeptide is an endogenous binding domain.

57. The macromolecule or pair of macromolecules of claim 56, wherein the endogenous binding domain is a ligand of the effector ligand or a fragment thereof.

58. The macromolecule or pair of macromolecules of claim 56, wherein the endogenous binding domain is a viral binding protein or a fragment thereof.

59. The macromolecule or pair of macromolecules of any one of claims 1-50, wherein the second binding domain comprises an oligonucleotide that specifically binds the effector ligand.

60. The macromolecule or pair of macromolecules of claim 59, wherein the oligonucleotide is a nucleic acid aptamer.

61. The macromolecule or pair of macromolecules of claim 60, wherein the nucleic acid aptamer is a DNA aptamer.

62. The macromolecule or pair of macromolecules of any one of claims 1-50, wherein the first binding domain, FBD1, or FBD2 comprises a chemical molecule that specifically binds the disease signature ligand.

63. The pair of macromolecules of any one of claims 5 and 7-62, wherein the second binding domain of the first member of the pair of macromolecules is a first portion of a binding moiety and the second binding domain of the second member of the pair of macromolecules is a second portion of the binding moiety.

64. The pair of macromolecules of any one of claims 5 and 7-62, wherein the second binding domain of the first member of the pair of macromolecules specifically binds to a first component of a heterodimeric receptor and the second binding domain of the second member specifically binds to a second component of the heterodimeric receptor.

65. The pair of macromolecules of any one of claims 5 and 7-62, wherein the second binding domain of the first member of the pair of macromolecules is a first component of a dimeric moiety and the second binding domain of the second member is a second component of a dimeric moiety.

66. The pair of macromolecules of any one of claims 5 and 7-62, wherein the second binding domain of the first member of the pair of macromolecules is a first fragment of a polypeptide chain and the second binding domain of the second member is a second fragment of the polypeptide chain.

67. The pair of macromolecules of claim 66, wherein the polypeptide chain is a hormone, a cytokine, or a growth factor.

68. The pair of macromolecules of any one of claims 5 and 7-67, wherein the second binding domain of the first member of the pair of macromolecules and the second binding domain of the second member of the pair of macromolecules have been engineered to have reduced affinity for one another.

69. The macromolecule or pair of macromolecules of any one of claims 1-68, wherein the macromolecule comprises a reporter moiety.

70. The macromolecule or pair of macromolecules of claim 69, wherein the reporter moiety is an affinity tag, a fluorescent marker, a radioactive marker, or a chromogenic marker.

71. The macromolecule or pair of macromolecules of claim 70, wherein the affinity tag is a FLAG affinity tag or the chromogenic marker is luciferase or beta-lactamase.

72. The macromolecule or pair of macromolecules of any one of claims 1-71, further comprising one or more linker domains.

73. The macromolecule or pair of macromolecules of claim 72, wherein the one or more linker domains are peptide linkers.

74. The macromolecule or pair of macromolecules of claim 73, wherein the peptide linkers comprise GS linkers.

75. The macromolecule or pair of macromolecules of claim 74, wherein the GS linkers comprise GS(GₙS)ₘ linkers.

76. The macromolecule or pair of macromolecules of claim 74, wherein the GS linkers comprise (GₙS)ₘ linkers.

77. The macromolecule or pair of macromolecules of any one of claims 1-31, 36-58, and 63-76, wherein the macromolecule is a polypeptide.

78. The macromolecule or pair of macromolecules of any one of claims 1-77, wherein the biological sample is an extract, fluid, fraction, cell, tissue, or subject.

79. The macromolecule or pair of macromolecules of any one of claims 1-78, wherein the macromolecule or one or both members of pair of macromolecules comprises a leader sequence.

80. The macromolecule or pair of macromolecules of claim 79, wherein the leader sequence comprises a secretion signal.

81. The macromolecule or pair of macromolecules of any one of claims 1-80, wherein the macromolecule or one or both members of pair of macromolecules comprises a half-life extension moiety.

82. The macromolecule or pair of macromolecules of claim 81, wherein the half-life extension moiety is an Fc domain or a fragment thereof, an albumin domain or a fragment thereof, or polyethylene glycol (PEG) or a modified derivative thereof.

83. A nucleic acid encoding the macromolecule of any one of claims 1-4 and 9-82.

84. A pair of nucleic acids encoding the pair of macromolecules of any one of claims 5-83.

85. The nucleic acid or pair of nucleic acids of claim 83 or 84, wherein the nucleic acid is an RNA or a DNA.

86. The nucleic acid or pair of nucleic acids of any one of claims 83-85, wherein the nucleic acid is formulated with a delivery platform.

87. The nucleic acid or pair of nucleic acids of claim 86, wherein the delivery platform is a lipid-based carrier or a vector delivery system.

88. The nucleic acid or pair of nucleic acids of claim 87, wherein the lipid-based carrier is a lipid nanoparticle (LNP).

89. The nucleic acid or pair of nucleic acids of claim 87, wherein the vector delivery system comprises or is derived from an adenovirus, an anellovirus, an AAV, or a lentivirus.

90. A nucleic acid encoding the macromolecule of any one of claims 1-4 and 9-82, wherein the nucleic acid is formulated with a carrier.

91. A pair of nucleic acids encoding the pair of macromolecules of any one of claims 5-82, wherein the pair of nucleic acids is formulated with a carrier.

92. The nucleic acid or pair of nucleic acids of claim 90 or 91, wherein the nucleic acid is an RNA or a DNA.

93. The nucleic acid or pair of nucleic acids of claim 90 or 91, wherein the carrier is a lipid-based carrier.

94. The nucleic acid or pair of nucleic acids of claim 93, wherein the lipid-based carrier is a LNP.

95. A vector comprising the nucleic acid of claim 83 or 85.

96. A vector or pair of vectors comprising the pair of nucleic acids of claim 84 or 85.

97. The vector or pair of vectors of claim 95 or 96, wherein the vector or pair of vectors is formulated with a carrier.

98. A host cell comprising the nucleic acid or pair of nucleic acids of any one of claims 83-94 or the vector or pair of vectors of any one of claims 95-97.

99. A multimer comprising two or more of the macromolecule of any one of claims 1-4 and 9-82.

100. The multimer of claim 99, wherein each of the macromolecule comprises the same amino acid sequence.

101. A multimer comprising at least one copy of each of the pair of macromolecules of any one of claims 5-82.

102. The macromolecule complex or macromolecule of any one of claims 1-82 or the nucleic acid or pair of nucleic acids of any one of claims 83-94, wherein the macromolecule complex, macromolecule, nucleic acid, or pair of nucleic acids is more than 95% pure.

103. The macromolecule complex or macromolecule of any one of claims 1-82 or the nucleic acid or pair of nucleic acids of any one of claims 83-94, wherein the macromolecule complex, macromolecule, nucleic acid, or pair of nucleic acids is manufactured according to the U.S. Food and Drug Administration (FDA)'s Good Manufacturing Practice (GMP), Good Clinical Practice (GCP), and/or Good Laboratory Practice (GLP) standards.

104. A method comprising providing the macromolecule or pair of macromolecules of any one of claims 1-82, the nucleic acid or pair of nucleic acids of any one of claims 83-94, or the multimer of any one of claims 99-101, access to a cell.

105. A method of modulating the state of a cell, comprising providing the macromolecule or pair of macromolecules of any one of claims 1-82, the nucleic acid or pair of nucleic acids of any one of claims 83-94, or the multimer of any one of claims 99-101, access to the cell, thereby modulating the state of the cell.

106. A method of inducing a cellular effector function in a cell, comprising providing the macromolecule or pair of macromolecules of any one of claims 1-82, the nucleic acid or pair of nucleic acids of any one of claims 83-94, or the multimer of any one of claims 99-101, access to the cell, thereby inducing the cellular effector function in the cell.

107. The method of claim 105 or 106, wherein the cell is in a subject and the macromolecule, nucleic acid, or multimer is administered in a therapeutically effective amount.

108. The method of claim 107, wherein the subject has, or is suspected of having, a disease or disorder characterized by abnormal levels of the disease signature target, optionally wherein the subject was previously determined to have abnormal levels of the disease signature target.

109. A method of determining the state of a cell, comprising providing the macromolecule or pair of macromolecules of any one of claims 69-71, a multimer thereof, or a nucleic acid or pair of nucleic acids encoding the same access to the cell, and detecting the presence of the reporter domain, thereby determining the state of the cell.

110. A method of inducing a cellular effector function in a cell, the method comprising contacting the cell with a macromolecule comprising a first binding domain linked to a second binding domain, wherein:
(a) the first binding domain specifically binds a disease signature ligand in a biological sample comprising the cell; and
(b) the second binding domain specifically binds an effector ligand in the biological sample and induces the cellular effector function upon binding to the effector ligand;
wherein the macromolecule is capable of forming a multimer in the presence of the disease signature ligand, and wherein induction of the effector function by the macromolecule is conditional upon each member of the multimer binding the disease signature ligand.

111. A method of inducing a cellular effector function in a cell, the method comprising contacting the cell with a pair of macromolecules, each independently comprising a first binding domain linked to a second binding domain, wherein:
(a) the first binding domain of each macromolecule specifically binds a disease signature ligand in a biological sample comprising the cell;
(b) the second binding domain of a first member of the pair of macromolecules specifically binds a first effector ligand in the biological sample; and
(c) the second binding domain of a second member of the pair of macromolecules specifically binds a second effector ligand in the biological sample;
wherein the second binding domain of the first member of the pair of macromolecules and the second binding domain of the second member of the pair of macromolecules induce the cellular effector function upon binding to the first effector ligand and the second effector ligand;
wherein the pair of macromolecules is capable of forming a heteromultimer in the presence of the disease signature ligand, and wherein induction of the effector function by the macromolecule is conditional upon each member of the heteromultimer binding the disease signature ligand.

112. A pair of macromolecules, each comprising a first binding domain linked to a second binding domain, wherein:
(a) the first binding domain of a first member of the pair of macromolecules specifically binds a first moiety of a disease signature ligand in a biological sample;
(b) the first binding domain of a second member of the pair of macromolecules specifically binds a second moiety of the disease signature ligand in the biological sample; and
(c) the second binding domain of each macromolecule specifically binds an effector ligand in the biological sample and induces a cellular effector function upon binding to the effector ligand;
wherein the macromolecule is capable of forming a heteromultimer in the presence of the disease signature ligand, and wherein induction of the effector function by the macromolecule is conditional upon each member of the heteromultimer binding the disease signature ligand.

113. A set of three macromolecules, each comprising a first binding domain linked to a second binding domain, wherein:
(a) the first binding domain of each macromolecule specifically binds a disease signature ligand in a biological sample;
(b) the second binding domain of a first member of the set of macromolecules specifically binds a first effector ligand in the biological sample;
(c) the second binding domain of a second member of the set of macromolecules specifically binds a second effector ligand in the biological sample; and
(d) the second binding domain of a third member of the set of macromolecules specifically binds a third effector ligand in the biological sample;
wherein the second binding domain of the first, second, and third members of the pair of macromolecules induce a cellular effector function upon binding to the first, second, and third effector ligands;
wherein the set of macromolecules is capable of forming a heterotrimer in the presence of the disease signature ligand, and wherein induction of the effector function by the macromolecule is conditional upon each member of the heterotrimer binding the disease signature ligand.

114. The set of three macromolecules of claim 113, wherein the disease signature ligand is trimeric.

115. The macromolecule or pair of macromolecules of any one of claims 1 and 5-7, wherein binding of the second binding domain to the effector ligand is conditional upon each member of the multimer binding the disease signature ligand.

116. The macromolecule or pair of macromolecules of any one of claims 1 and 5-7, wherein binding of the second binding domain to the effector ligand is not conditional upon each member of the multimer binding the disease signature ligand.

117. A plurality of macromolecules, each independently comprising a first binding domain linked to a second binding domain, wherein:
(a) the first binding domain of each macromolecule specifically binds a disease signature ligand in a biological sample; and
(b) the second binding domain of each macromolecule specifically binds an effector ligand in the biological sample;
wherein the plurality of macromolecules is capable of forming a multimer in the presence of the disease signature ligand, and wherein induction of the effector function by the macromolecule is conditional upon each member of the multimer binding a disease signature ligand.

118. The plurality of macromolecules of claim 117, wherein the multimer is a homodimer, a homotrimer, or a homotetramer.

119. The plurality of macromolecules of claim 117, wherein the multimer is a heterodimer, a heterotrimer, or a heterotetramer.

120. The plurality of macromolecules of any one of claims 117-119, wherein at least one macromolecule comprises a disease signature ligand that is a protein, a peptide, or a small molecule.

121. The plurality of macromolecules of any one of claims 117-120, wherein binding of the second binding domain to the effector ligand is conditional upon each member of the multimer binding the disease signature ligand.

122. The plurality of macromolecules of any one of claims 117-120, wherein binding of the second binding domain to the effector ligand is not conditional upon each member of the multimer binding the disease signature ligand.


SEQUENCE LISTING (33 sequences)

SEQ ID NO: 1 - protein, 122 aa, synthetic construct
DVQLVQSGAEVKKPGASVKVSCKASGFTFDNYAMHWVRQAPGQGLEWIGYISWNSGDINYADSVKGRFTITTDKSTSTAYMELSSLRSEDTATYYCARDAGFGEFHYGLDVWGQGTTVTVSS

SEQ ID NO: 2 - protein, 107 aa, synthetic construct
DIVLTQSPATLSLSPGERATLSCRASQGISSAMNWYQQKPGKAPKRWIYDASKVASGVPARFSGSGSGTDYSLTINSLEAEDAATYYCQQFNSYPWTFGGGTKVEIK

SEQ ID NO: 3 - protein, 27 aa, synthetic construct
PKKKIQLHAEHALYDALMILNIVKTNS

SEQ ID NO: 4 - protein, 76 aa, synthetic construct
TNSPPAEEKLEDYAFNFELILEEIARLFESGDQKDEAEKAKRMKEWMKRIKTTASEDEQEEMANAIITILQSWIFS

SEQ ID NO: 5 - protein, 249 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVL

SEQ ID NO: 6 - protein, 127 aa, synthetic construct
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSS

SEQ ID NO: 7 - protein, 126 aa, synthetic construct
QVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSS

SEQ ID NO: 8 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 9 - protein, 106 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 10 - protein, 122 aa, synthetic construct
EVQLVESGGGLVQPGRSLRLSCATSGFTFDNYAMYWVRQAPGKGLEWVSGISWNSGDIGYADSVKGRFTISRDNAKNSLYLQMNSLRAEDTALYYCARDAGFGEFHYGLDVWGQGTTVTVSS

SEQ ID NO: 11 - protein, 108 aa, synthetic construct
AIQLTQSPSSLSASVGDRVTITCRASQGISSALAWYQQKPGKVPKLLIYDASSLESGVPSRFSGSGSGTDFTLTISSLQPEDFATYYCQQFNSYPWTFGQGTKVEIKR

SEQ ID NO: 12 - protein, 248 aa, synthetic construct
QVQLVQSGAEVKKPGASVKVSCKASGYEFTSYWIHWVRQAPGQGLEWMGNISPNSGSANYNEKFKSRVTMTRDTSTSTVYMELSSLRSEDTAVYYCAREGPYSYYPSREYYGSDLWGQGTLVTVSSGGGGSGGGGSGGGGSEIVLTQSPATLSLSPGERATLSCRASQSISNNLHWYQQKPGQAPRLLIYYTSRSVSGIPARFSGSGSGTDFTLTISSLEPEDFAVYYCGQNNEWPEVFGGGTKVEIK

SEQ ID NO: 13 - protein, 120 aa, synthetic construct
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 14 - protein, 108 aa, synthetic construct
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 15 - protein, 25 aa, synthetic construct
MGVLLTQRTLLSLVLALLFPSMASM

SEQ ID NO: 16 - protein, 16 aa, synthetic construct
MKCLLYLAFLFIGVNC

SEQ ID NO: 17 - protein, 21 aa, synthetic construct
METDTLLLWVLLLWVPGSTGD

SEQ ID NO: 18 - protein, 19 aa, synthetic construct
MGWSCIILFLVATATGVHS

SEQ ID NO: 19 - protein, 17 aa, synthetic construct
MRAWIFFLLCLAGRALA

SEQ ID NO: 20 - protein, 21 aa, synthetic construct
MWWRLWWLLLLLLLLWPMVWA

SEQ ID NO: 21 - protein, 22 aa, synthetic construct
MDMRVPAQLLGLLLLWLRGARC

SEQ ID NO: 22 - protein, 16 aa, synthetic construct
MPLLLLLPLLWAGALA

SEQ ID NO: 23 - protein, 23 aa, synthetic construct
MDAMKRGLCCVLLLCGAVFVSPS

SEQ ID NO: 24 - protein, 18 aa, synthetic construct
MAFLWLLSCWALLGTTFG

SEQ ID NO: 25 - protein, 15 aa, synthetic construct
MNLLLILTFVAAAVA

SEQ ID NO: 26 - protein, 20 aa, synthetic construct
MYRMQLLSCIALSLALVTNS

SEQ ID NO: 27 - protein, 17 aa, synthetic construct
MGVKVLFALICIAVAEA

SEQ ID NO: 28 - protein, 16 aa, synthetic construct
MKWVTFISLLFSSAYS

SEQ ID NO: 29 - protein, 16 aa, synthetic construct
MKTIIALSYIFCLVLG

SEQ ID NO: 30 - protein, 24 aa, synthetic construct
MALWMRLLPLLALLALWGPDPAAA

SEQ ID NO: 31 - protein, 16 aa, synthetic construct
MKPIFLVLLVVTSAYA

SEQ ID NO: 32 - protein, 126 aa, synthetic construct
QVQLVESGGGLVQPGGSLRLSCAASGFSLDYYGVGWFRQAPGKEREGVSCISSSDGDTYYADSVKGRFTISRDNAKNTVYLQMNSLKPEDTAVYYCATDLSDYGVCSRWPSPYDYWGQGTQVTVSS

SEQ ID NO: 33 - protein, 125 aa, synthetic construct
AVQLVDSGGGLVQAGGSLRLSCAASGRFTSSSPMGWFRQAPGKEREFVAAISGRSGNTYYADSVKGRFTISRDNAKNTVYLQMNSLKPEDTAVYYCAAERVGLLLTVVAEGYDYWGQGTQVTVSS